SEQUENCE LISTING (29 sequences)

SEQ ID NO: 1 - DNA, 6943 bp, Artificial Sequence
  pAAV COL4A5 c.1871G>A
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagcccaggtttaccaggcctcccagggaatatagggcctatgggtccccctggtttcggccctccaggcccagtaggtgaaaaaggcatacaaggtgtggcaggtcgaaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccctagcggattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttcgtctctgtcgcctggagggccgaaatcagggggtctgcgagacggaattctgcaatcgacggtaccacgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgccgcgggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctggagggccgaaatcagggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - DNA, 7439 bp, Artificial Sequence
  PX551 COL4A5 c.1871G>A SpCas9
ggcgtcgggcgacctttggtcgcccggcctcagtgagcgagcgagcgcgcagagagggagtggccaactccatcactaggggttcctgcggcctctagaaagcttccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcaccggcctggagggccgaaatcagggggccggtgccaccatgtacccatacgatgttccagattacgcttcgccgaagaaaaagcgcaaggtcgaagcgtccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccg

SEQ ID NO: 3 - DNA, 6707 bp, Artificial Sequence
  pAAV MECP2 c.473C>T
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggattgcgtacttcgaaaaggtaggcgacacatccctggaccctaatgattttgacttcacggtaacggggagagggagcccctcccggcgagagcagaaaccacctaagaagcccaaatcgtcgacccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcggattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttcgtctctgtcggattttgacttcatggtaactggtctgcgagacggaattctgcaatcgacggtaccacgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgccgcgggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggattttgacttcatggtaacgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 4 - DNA, 7427 bp, Artificial Sequence
  PX551 MECP2 c.473C>T SpCas9
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaaagcttagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccgggattttgacttcatggtaactggccggtgccaccatgtacccatacgatgttccagattacgcttcgccgaagaaaaagcgcaaggtcgaagcgtccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 6208 bp, Artificial Sequence
  pAAV LRRK2 c.6055G>A
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaattttaccctgtatcccaatgctgccatcattgcgaagattgcggactacgggatcgcacagtactgctgcaggatgggaataaagacatcagagggcacctcgacggtatcgttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccctagcggattcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttcgtctctgtcgagcagtactgtgcgatcgagtagtctgcgagacggaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaatcgaattcctgcagcccgggggatccactaggggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagcagtactgtgcgatcgaggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtaccctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 6 - DNA, 8239 bp, Artificial Sequence
  PX551 LRRK2 c.6055G>A SaCas9
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagtcgaggtcgacggtatcgataagcttgattatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagcttggatccagcagtactgtgcgatcgagtagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgactactcgatcgcacagtactgctccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 7 - DNA, 101 bp, Artificial Sequence
  COL4A5 Donor
cccaggtttaccaggcctcccagggaatatagggcctatgggtccccctggtttcggccctccaggcccagtaggtgaaaaaggcatacaaggtgtggcag

SEQ ID NO: 8 - DNA, 120 bp, Artificial Sequence
  MECP2 Donor
gattgcgtacttcgaaaaggtaggcgacacatccctggaccctaatgattttgacttcacggtaacggggagagggagcccctcccggcgagagcagaaaccacctaagaagcccaaatc

SEQ ID NO: 9 - DNA, 100 bp, Artificial Sequence
  LRRK2 Donor
ttttaccctgtatcccaatgctgccatcattgcgaagattgcggactacgggatcgcacagtactgctgcaggatgggaataaagacatcagagggcacc

SEQ ID NO: 10 - DNA, 577 bp, Artificial Sequence
  CMV promoter
cgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 11 - DNA, 945 bp, Artificial Sequence
  mCherry cds
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttcgtctctgtcggattttgacttcatggtaactggtctgcgagacggaattctgcaatcgacggtaccacgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctga

SEQ ID NO: 12 - DNA, 20 bp, Artificial Sequence
  COL4A5 target sequence
cctggagggccgaaatcagg

SEQ ID NO: 13 - DNA, 20 bp, Artificial Sequence
  MECP2 target sequence
gattttgacttcatggtaac

SEQ ID NO: 14 - DNA, 20 bp, Artificial Sequence
  LRRK2 target sequence
agcagtactgtgcgatcgag

SEQ ID NO: 15 - DNA, 719 bp, Artificial Sequence
  GFP cds
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagta

SEQ ID NO: 16 - DNA, 249 bp, Artificial Sequence
  Human U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 17 - DNA, 96 bp, Artificial Sequence
  COL4A5 c.1871G>A sgRNA cds
cctggagggccgaaatcagggttttagagctagaaatagcaagttaaaataaggctagtccgttatcaacttgaaaaagtggcaccgagtcggtgc

SEQ ID NO: 18 - DNA, 96 bp, Artificial Sequence
  MECP2 c.473C>T sgRNA cds
gattttgacttcatggtaacgttttagagctagaaatagcaagttaaaataaggctagtccgttatcaacttgaaaaagtggcaccgagtcggtgc

SEQ ID NO: 19 - DNA, 96 bp, Artificial Sequence
  LRRK2 c.6055G>A sgRNA cds
agcagtactgtgcgatcgaggttttagagctagaaatagcaagttaaaataaggctagtccgttatcaacttgaaaaagtggcaccgagtcggtgc

SEQ ID NO: 20 - DNA, 529 bp, Woodchuck hepatitis virus
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctctt

SEQ ID NO: 21 - DNA, 229 bp, Mus musculus
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 22 - DNA, 4101 bp, Streptococcus pyogenes
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 23 - DNA, 4101 bp, Staphylococcus aureus; identical to SEQ ID NO: 22

SEQ ID NO: 24 - DNA, 20 bp, Artificial Sequence
  COL4A5 c.1871G>A sgRNA cds guide sequence
cctggagggccgaaatcagg

SEQ ID NO: 25 - DNA, 76 bp, Artificial Sequence
  COL4A5 c.1871G>A sgRNA cds - scaffold sequence
gttttagagctagaaatagcaagttaaaataaggctagtccgttatcaacttgaaaaagtggcaccgagtcggtgc

SEQ ID NO: 26 - DNA, 20 bp, Artificial Sequence
  MECP2 c.473C>T sgRNA cds - guide sequence
gattttgacttcatggtaac

SEQ ID NO: 27 - DNA, 76 bp, Artificial Sequence; identical to SEQ ID NO: 25
  MECP2 c.473C>T sgRNA cds - scaffold sequence

SEQ ID NO: 28 - DNA, 20 bp, Artificial Sequence
  LRRK2 c.6055G>A sgRNA cds - guide sequence
agcagtactgtgcgatcgag

SEQ ID NO: 29 - DNA, 76 bp, Artificial Sequence; identical to SEQ ID NO: 25
  LRRK2 c.6055G>A sgRNA cds - scaffold sequence

(19) United States
(12) Patent Application Publication
(10) Pub. No.: US 2023/0287368 A1
(43) Pub. Date: Sep. 14, 2023

(54) CRISPR-CAS SYSTEM FOR GENE THERAPY

(71) Applicants: UNIVERSITA' DEGLI STUDI DI SIENA, Siena (IT); AZIENDA OSPEDALIERA UNIVERSITARIA SENESE (AOUS), Siena (IT)

(72) Inventors: Alessandra RENIERI, Siena (IT); Silvestro CONTICELLO, Firenze (IT); Anna Maria PINTO, Veglie (Lecce) (IT); Ilaria MELONI, Murlo (Siena) (IT); Sergio DAGA, Novoli (Lecce) (IT); Francesco DONATI, Arezzo (IT); Susanna CROCI, Siena (IT); Diego LOPERGOLO, Roma (IT)

(21) Appl. No.: 17/415,642

(22) PCT Filed: Dec. 17, 2019
(86) PCT No.: PCT/EP2019/085659

§ 371 Date: Jun. 17, 2021

(30) Foreign Application Priority Data
Dec. 19, 2018 (IT) 102018000020230

(51) Int. Cl. C12N 9/22; C12N 15/10; C12N 15/86

(52) U.S. Cl. CPC C12N 9/22; C12N 15/102; C12N 15/86; C12N 2310/20; C12N 2750/14143

(57) ABSTRACT

There is described a genome editing system, more particularly a dual vector CRISPR-Cas system suitable for gene editing-mediated correction of a mutant genomic target sequence in a target cell. There are further described a set of two viral particles and a host eukaryotic cell comprising the CRISPR-Cas system of the invention as well as the therapeutic use of this system, particularly for the treatment of genetic diseases.

### FIELD OF THE INVENTION

The present invention relates to the field of genome editing, in particular to a CRISPR-Cas system targeting a mutant genomic target sequence carrying one or more mutations in a target cell as well as to a viral particle comprising such system.

### SEQUENCE LISTING

A sequence listing is submitted as an ASCII computer readable text file entitled E0134890 with a creation date of 17 Dec. 2021 and a size of 76,584 bytes or 77,824 bytes on disk, submitted pursuant to 37 CFR 1.821(c) and/or 37 CFR 1.821(e), which is fully incorporated by reference herein in its entirety.

### BACKGROUND OF THE INVENTION

Monogenic diseases arise from mutations in a single gene. Although relatively rare as single entities, these diseases collectively affect millions of people worldwide. So far, there are no curative treatments available for the majority of monogenic disorders and the therapeutic approaches have been essentially based on the symptomatic treatment of the patients, with the aim of improving patients' quality of life and life expectancy.

Among monogenic disorders, Alport syndrome (ATS) is a hereditary kidney disease characterized by progressive renal failure, variable sensorineural hearing loss and ocular anomalies. Such disease represents a heterogeneous condition resulting from mutations in COL4A3, COL4A4 and COL4A5 genes, encoding type IV collagen. Currently, a curative therapy for ATS is not available, and the functional disruptions of podocytes can be only partially recovered by symptomatic treatments, with dialysis or kidney transplant being the only possible final intervention. When patients with Alport syndrome receive renal transplant, posttransplant antiglomerular basement membrane (anti-GBM) nephritis may occur causing transplant failure, thereby limiting the applicability of this therapeutic option in those patients for which a compatible donor is not available.

An extremely debilitating genetic disease is Pompe Disease or Glycogen Storage Disease Type II, which is an autosomal recessive disorder caused by mutations in the GAA gene. These mutations prevent the acid alpha-glucosidase enzyme from breaking down glycogen effectively, resulting in this sugar building up to toxic levels in lysosomes. The accumulation of glycogen damages organs and tissues throughout the entire body, particularly the heart, the skeletal muscles, the liver and the nervous system leading to the progressive signs and symptoms of Pompe disease. The classic form of infantile-onset Pompe disease begins within a few months of birth. Infants with this disorder typically experience muscle weakness (myopathy), poor muscle tone (hypotonia), an enlarged liver (hepatomegaly), and heart defects. If untreated, this form of Pompe disease leads to death from heart failure within the first year of life. The late-onset type of Pompe disease may not become apparent until later in childhood, adolescence, or adulthood and it is usually milder than the infantile-onset form. Most individuals with late-onset Pompe disease experience progressive muscle weakness, especially in the legs and the trunk, including the muscles that control breathing. As the disorder progresses, breathing problems can lead to respiratory failure. An enzyme replacement therapy (ERT) (FDA approved Myozyme and Lumizyme) is currently available for the treatment of Pompe disease, yet it is a lifelong treatment and it is not completely resolutive since it is difficult to attain effective concentrations of the recombinant enzyme in target tissues in the central nervous system. In particular, Myozyme is not a single shot therapy, requires a life-long administration since it is given as an infusion once every two weeks, and clinical trials carried out so far are not conclusive about the benefits of the treatment in the late-onset form. In infantile Pompe disease patients, the glycogen accumulates in the brainstem motor and sensory neurons, interneurons, and motor neurons. Precocious systemic absence of the GAA gene in the mouse induces a complex neuropathological cascade in the spinal cord indicating a widespread neuropathology. The emergent neurologic phenotype in some patients and the frequent persistence of bulbar muscular weakness may be attributed to CNS lesions, uncorrected by ERT because of the blood-brain-barrier.

Rett syndrome and related disorders represent the second most common cause of intellectual disability in females with an estimated incidence of 1:10,000 live births. This disorder is primarily caused by mutations in the MECP2, FOXG1, or CDKL5 genes but a number of new genes have been recently reported and some more are probably still waiting identification, as a variable percentage of patients result negative to genetic screening for the known genes. Currently, there are no treatments or disease-modifying therapies available for Rett syndrome. Historically, the primary risk anticipated for viral vector-mediated MECP2 gene transfer in vivo has been toxicity related to over-expression of exogenous MeCP2 within transduced neurons, outlining the importance of a fine native regulation. According to Collins A. L. et al, “Mild overexpression of MeCP2 causes a progressive neurological disorder in mice”; (2004, Hum. Mol. Genet., 13(21):2679-2689) transgenic mice overexpressing MeCP2 suffered from tremors, weight loss, disheveled appearance, swaying, seizures, lesions, hypoactivity, gait abnormalities, and/or early death.

Parkinson's disease (PD) is the second most common neurodegenerative disorder worldwide, clinically characterized by motor and non-motor symptoms mostly due to dopaminergic neurons death in the substantia nigra. Although most cases of PD are sporadic, rare familial forms are caused by mutations in single genes. Thirteen genes have been described as possibly responsible for hereditary PD, and six of them (LRRK2, VPS35, PRKN, DJ-1, SNCA, PINK1) have been linked with an autosomal dominant or recessive form of PD (Kalinderi K. et al, “The genetic background of Parkinson's disease: current progress and future prospects”; (2016) Acta Neurol Scand., 134(5):314-326). Besides monogenic forms, a number of PD risk alleles have been identified. For instance, heterozygous variations in the GBA1 gene, coding for the lysosomal enzyme glucocerebrosidase, confer five- to seven-fold increased risk, resulting as the most common genetic risk factor for PD. At present, there is no cure available for the treatment of PD. The pharmacological therapies for patients affected by this disease, based on the administration of levodopa or dopamine agonists, are purely symptomatic and many patients become resistant to levodopa treatment with disease progression and longer disease duration.

In the last decade, genome editing has been extensively exploited for the treatment of monogenic disorders and clinical trials for several diseases are currently ongoing. Most strategies rely on the so-called gene replacement therapy, which involves the insertion of an additional copy of the gene associated with the disease somewhere in the genome, under the control of a non-specific promoter, and are based on the principle that the increased gene dosage may be curative for the target disease. However, in several monogenic disorders it has been observed a dominant negative effect of the protein encoded by the mutated gene, thereby reducing significantly the efficacy of the reintroduction of the wild-type protein. Moreover, in a number of disorders such as e.g. Rett syndrome, gene overexpression has been shown to be as detrimental as haploinsufficiency, making it extremely difficult to achieve a fine-tuned modulation of gene expression levels in a gene-replacement therapy.

Gene therapy approaches relying on the stable correction of the mutant allele/s appear a more promising alternative for the cure of monogenic diseases. In these approaches, the mutant gene sequence is snipped out from the genome and fully replaced by the wild-type sequence, which can therefore maintain its native regulation.

Of recently developed ‘gene editing’ technologies, the “Clustered Regularly Interspersed Short Palindromic Repeat (CRISPR)-CRISPR associated (Cas) (CRISPR-Cas)” system has brought considerable progress to the field. This technology enables introducing double-strand breaks into the genome with high specificity in order to mediate the modification of selected genomic regions (Luther D. C., et al, “Delivery Approaches for CRISPR/Cas9 Therapeutics in-vivo: Advances and Challenges”; (2018) Expert Opin Drug Deliv. 15(9):905-913). Thanks to high flexibility, the CRISPR-Cas system may be adapted to various needs, including the inactivation of a specific gene (Knock-out) or the replacement of a mutant gene sequence with the wild type counterpart (Knock-in). More specifically, the CRISPR-Cas system acts by inducing Double-Strand Breaks (DSB) in the genomic DNA by employing a short RNA sequence, the so-called RNA guide (gRNA), which drives an endonuclease enzyme towards a specific target site on the genome. Among employed endonucleases, the most common is the Cas9 nuclease from Streptococcus pyogenes (SpCas9). Typically, a gRNA molecule is made up of two parts: a nucleotide sequence complementary to the target DNA (“guide nucleotide sequence”), and a nucleotide sequence which serves as a binding scaffold for the endonuclease enzyme (“scaffold nucleotide sequence”). This molecule is also referred to as “single guide RNA” (sgRNA) to differentiate such gRNA from the guide RNA format existing in nature as two separate “guide” and “scaffold” molecules. Cas9-induced DBS are repaired by the cellular repair DNA mechanisms, and the output of CRISPR-Cas activity depends on the specific DBS repair mechanism. Repair by non-homologous end joining (NHEJ) does not require a repair template and leads occasionally to the generation of small insertions/deletions (indel) at the cleavage site, thereby resulting in the disruption of the target gene and the abrogation of its expression (knock-out). Alternatively, if a donor DNA with homology to the targeted locus is supplied, DSB can be repaired by homology-directed repair (HDR), allowing for precise replacement mutations to be made (knock-in). When the sgRNA is designed to specifically recognize a mutant allele of a gene and in presence of a donor DNA harboring the sequence of the wild type allele, HDR machinery results in the correction of the gene mutation. Of note, HDR is effective in both dividing and non-dividing cells, such as terminally differentiated neurons or muscle cells, and therefore gene correction may be achieved also in the treatment of disorders affecting tissues with limited regeneration/renewal capacity, including the CNS.

However, in vivo applications of the CRISPR-Cas system, particularly as a therapeutic tool, are still hampered by the inefficiency of precise base editing and the high frequency of Cas9-induced genomic cleavages at sites that differ from the intended genomic target. Such off-target effects occur as Cas9 can tolerate up to 5 base mismatches between the gRNA and the target sequence in the genome, and long term expression of Cas9 increases the frequency of these effects.

International patent application WO2015070083 discloses the use in a CRISPR-Cas system of a gRNA molecule, referred to as “governing gRNA molecule”, in addition to the gRNA targeting the specific sequence of interest. Such “governing gRNA” acts as negative regulator of Cas9 activity by targeting the coding sequence for this enzyme, or, alternatively, by targeting a control region, e.g., a promoter, that regulates the expression of the Cas9 coding sequence.

International patent application WO2015089354 describes also a self-inactivating CRISPR-Cas composition comprising a first CRISPR complex directed towards a target DNA and a second CRISPR complex directed towards a polynucleotide encoding a component of the CRISPR-Cas system, whereby the activity of the first CRISPR-Cas system may be controlled and diminished over a period of time.

International patent application WO2018069474 discloses a Self-Limiting Cas9 circuitry for Enhanced Safety (SLiCES,) which consists of an expression unit for the Streptococcus pyogenes Cas9 (SpCas9) along with a first gRNA towards a specific genomic locus and a second Cas9 self-targeting gRNA to switch off the nuclease activity. The SLiCES system is integrated into a lentiviral delivery system (lentiSLiCES) via circuit inhibition to avoid the leaky expression of SpCas9 during viral particle production. Such integration requires the introduction in the vector of an inducible promoter and/or an intron in the SpCas9 open reading frame.

International patent application WO2016176191 describes a dual AAV vector system for CRISPR-Cas9 mediated correction of genetic errors in human diseases, which targets proliferating progenitor and/or stem cells in developing tissues in order to populate these tissues with the gene-corrected cells. In the disclosed system, regulation of Cas9 activity is achieved through the dilution of Cas elements during cell proliferation.

Despite the efforts to reduce undesirable off-target effects, the approaches reported so far suffer from significant limitations primarily related to the structural complexity of the employed CRISPR-Cas systems, which require the use of specifically designed additional components, such as additional gRNA molecules, as well as the incorporation of further trans-acting factors and promoters in the construction of the delivery system. This is particularly problematic in the case of adeno-associated virus (AAV) vectors, because of their limited cloning capacity. Moreover, the avoidance of off targets effects through cell division-based dilution of Cas9 nuclease prevents the use of the CRISPR-Cas system for targeting non-replicative cells, thereby limiting its therapeutic application for treating disorders involving such cell types.

### SUMMARY OF THE INVENTION

Thus, there exists a need in the art for a CRISPR-Cas system which does not suffer from the drawbacks and limitations of the prior art.

In particular, there is a need for a CRISPR-Cas system capable of controlling its activity in order to abolish or minimize undesirable off-targets effects, without affecting system simplicity, reliability and accuracy, thereby enabling safely use of this system in therapeutic applications, particularly in gene therapy.

These and other needs are met by the CRISPR-Cas system and the set of two viral particles comprising the CRISPR-Cas system as described and claimed herein.

Another aspect of the present invention is an in vitro method for editing a mutant genomic target sequence in a target cell.

A further aspect of the invention is the CRISPR-Cas system of the invention and/or the viral particles of the invention for use as a medicament, in particular in gene therapy.

A still further aspect of the invention is a pharmaceutical composition comprising the CRISPR-Cas system of the invention or the set of two viral particles of the invention, and a pharmaceutically acceptable carrier, diluent and/or vehicle.

Other features and advantages of the present invention are defined in the appended claims which form an integral part of the description.

As disclosed in more detail below, the present invention provides a new self-limiting “Clustered Regularly Interspersed Short Palindromic Repeat (CRISPR)-CRISPR associated (Cas) (CRISPR-Cas)” system, which can achieve reliable controlled expression of Cas9 nuclease using a simple design and minimal elements.

The non-naturally occurring or engineered CRISPR-Cas system of the invention is a dual vector system designed to target a mutant genomic target sequence carrying one or more mutations in a target cell.

The first viral expression vector according to the invention comprises a nucleotide sequence encoding a guide RNA (gRNA), which comprises a scaffold nucleotide sequence capable of binding an endonuclease enzyme, and a guide nucleotide sequence capable of hybridizing to the mutant genomic target sequence.

As used herein, the term “guide RNA (gRNA)” is intended to refer to the gRNA format that combines both the “guide nucleotide sequence” and the “scaffold nucleotide sequence” into a single RNA molecule.

As used herein, the term “a nucleotide sequence encoding a guide RNA (gRNA)” is intended to refer to a nucleotide sequence which is transcribed into the gRNA molecule.

Preferably, the guide nucleotide sequence is 17 to 25 nucleotides in length, more preferably 20 to 21 nucleotides (Cencic R. et al, “Protospacer adjacent motif (PAM)-distal sequences engage CRISPR Cas9 DNA target cleavage” (2014) PLoS One 9(10): e109213).

The first viral expression vector of the invention further comprises a donor nucleotide sequence consisting of the wild type sequence of the mutant genomic target sequence targeted by the CRISPR-Cas system.

In the context of the present description, the term “wild-type” means the typical, naturally occurring form of a nucleotide sequence. Any form of a nucleotide sequence other than the wild-type is a “mutant” sequence.

According to the present invention, the donor nucleotide sequence located on the first viral expression vector and consisting of the wild-type sequence of the mutant genomic target sequence is suitable to function as template for homology directed repair (HDR) to replace at least one of the mutations in the target genomic sequence.

As indicated above, the CRISPR-Cas system of the invention comprises a second viral expression vector comprising a nucleotide sequence encoding an endonuclease enzyme.

An endonuclease enzyme, as that term is used herein, refers to a molecule that is capable to interact with the scaffold nucleotide sequence of the gRNA molecule and, in concert with the gRNA molecule, direct towards a specific site on the genome complementary to the guide nucleotide sequence of the gRNA. After binding, the endonuclease enzyme is capable of generating a double-strand break in the target genomic sequence.

Exemplary endonuclease enzymes suitable to be employed in the CRISPR-Cas system of the invention include, but are not limited to, the Cas9 nuclease and variants thereof which recognize different Protospacer Adjacent Motif (PAM) sequences. As it is known in the art, PAM motifs are sequences of 2-5 base pairs in length located in close proximity with the target sequence of a CRISPR-Cas system, which are required for Cas9 cleavage (Shah S. et al, “Protospacer recognition motifs Mixed identities and functional diversity (2013) RNA Biology 10(5): 891-899).

In a preferred embodiment, the endonuclease employed in the CRISPR-Cas system of the invention is selected from the group consisting of Streptococcus pyogenes Cas9 (SpCas9), SpCas9 D1135E variant, SpCas9 VRER variant, SpCas9 EQR variant, SpCas9 VQR variant, Staphylococcus aureus Cas9 (SaCas9), Sniper-Cas9 and SpCas9-HF1.

According to the present invention, both the nucleotide sequence encoding the gRNA, located on the first viral expression vector, and the nucleotide sequence encoding the endonuclease enzyme, located on the second viral expression vector, are operably linked to promoter sequences, which drive their transcription. Said promoter sequences are located upstream of the 5′ end of the gRNA-encoding nucleotide sequence and upstream of the 5′ end of the endonuclease-encoding nucleotide sequence, respectively.

The promoter may be any nucleic acid sequence showing transcriptional activity in a host cell, including constitutive promoters, inducible promoters and tissue-specific promoters.

In a preferred embodiment, the promoter sequence is selected from the group consisting of the Cytomegalovirus (CMV) promoter, the SV40 promoter, the MECP2 promoter, the U6 promoter, the H1 promoter, the chicken beta-actin promoter (CBA) and the human Elongation factor 1 alpha-subunit (EF1-alpha) promoter.

In another preferred embodiment, the donor nucleotide sequence located on the first expression vector which consists of the wild type sequence of the mutant genomic target sequence, is not operably linked to any promoter sequence and hence it cannot be expressed in a host cell.

According to the present invention, the second viral expression vector comprises additionally a target nucleotide sequence consisting of the mutant genomic sequence targeted by the CRISPR-Cas system. Preferably, the target nucleotide sequence consists of a short nucleotide sequence complementary to the nucleotide sequence of the gRNA and includes a PAM sequence. Said target sequence may be present on the viral expression vector in one or two copies. As one copy, the target nucleotide sequence is located between the 3′ end of the promoter sequence operably linked to the nucleotide sequence encoding the endonuclease enzyme and the 5′ end of said endonuclease-coding sequence. Alternatively, when the target nucleotide sequence is present on the viral expression vector in two copies, a first copy is located upstream of the 5′ end of the promoter sequence operably linked to the nucleotide sequence encoding the endonuclease enzyme and a second copy is located downstream of the 3′ end of said endonuclease-coding sequence.

As a consequence of the above described alternative configurations of the second viral expression vector of the invention, gRNA-directed endonuclease cleavage at the one or two copies of the target nucleotide sequence results either in a break in the intervening sequence between the promoter and the nucleotide sequence encoding the endonuclease, or in the excision of the entire endonuclease-coding unit, thereby preventing endonuclease expression. Hence, in the CRISPR-Cas system of the invention, the mutant-specific gRNA is capable to drive endonuclease-mediated genome editing as well as endonuclease-mediated auto-limiting control on enzyme expression.

Preferably, the one or two copies of the target nucleotide sequence in the second vector are from 10 to 30 nucleotides in length, more preferably from 15 to 25 nucleotides, for example 15, 16, 17, 18, 19, 20, 21, 22, 23, 24, 25 nucleotides in length.

In the second viral expression vector, each copy of the target nucleotide sequence includes at the 3′ end a PAM sequence. The precise sequence and length requirements for the PAM differ depending on the CRISPR endonuclease enzyme used. For example, a suitable PAM is 5′-NGG for Streptococcus pyogenes Cas9 (SpCas9) and SpCas9 D1135E variant (where N is any nucleotide), 5′-NGCG for SpCas9 VRER variant, 5′-NGAG for SpCas9 EQR variant, 5′-NGAN or NGNG for SpCas9 VQR variant, 5′-NNGRRT or NNGRR(N) for Staphylococcus aureus Cas9 (SaCas9) (where R is adenine or guanine), 5′-NG for Sniper-Cas9 and 5′-NGG for SpCas9-HF1.

In a preferred embodiment, the viral expression vector is an adenovirus or adeno-associated virus (AAV) vector, more preferably a serotype 2 adeno-associated viral vector (AAV2) or a serotype 9 adeno-associated viral vector (AAV9).

AAVs are particularly suitable as viral vectors in the present invention since they are non-integrating viruses which remain in an episomal state inside the cells, thus limiting the risk of insertional mutagenesis in the genome of infected cells. Numerous AAV serotypes are known in the art, which differ in their tissue tropism or the type of cell they infect. For instance, serotype 2 adeno-associated virus (AAV2) exhibits higher tropism towards the kidney parenchyma while serotype 9 adeno-associated virus (AAV9) is particularly suitable for targeting brain or neuronal cells since this virus is able to cross the blood-brain barrier.

In another embodiment, the viral expression vector in the CRISPR-Cas system of the invention may be a lentiviral vector, a retroviral vector or a parvovirus vector.

In order to facilitate encapsidation into a viral particle, the viral expression vector may comprise additional regulatory sequences. Examples of regulatory sequences serving this purpose include, but are not limited to, inverted terminal repeat (ITR) and Long Terminal Repeats (LTR) sequences, polyadenylation signal sequences, encapsidation signal (c) and packaging signal (w) sequences.

Preferably, either one or both the first and second viral expression vectors contain a reporter system, which is suitable for assessing the expression of said vectors into transfected target cells. Typically, a reporter system may contain one or more genes encoding, for example, an enzyme, such as e.g. the luciferase, or a fluorescent protein, such as e.g. the green fluorescent protein (GFP) or the red fluorescent protein mCherry, whose activities can be readily assayed subsequent to transfection. A preferred reporter system in the CRISPR-Cas system of the invention is the dual mCherry/GFP gene system.

According to the invention, the mutant genomic sequence targeted by the CRISPR-Cas system of the invention may be a coding sequence, such as e.g. a protein coding sequence, or a non-coding sequence, such as e.g., a regulatory element, for example a promoter, an enhancer, a silencer, a splicing donor or an acceptor sequence.

In one embodiment, the mutant genomic target sequence is selected from the group consisting of mutant COL4A5 gene, mutant COL4A3 gene, mutant COL4A4 gene, mutant GAA gene, mutant MECP2 gene, mutant FOXG1 gene, mutant CDKL5 gene, mutant LRRK2 gene, mutant VPS35 gene, mutant PRKN gene, mutant DJ-1 gene, mutant SNCA gene, mutant PINK1 gene and mutant GBA1 gene.

In another embodiment, the one or more mutations carried by the genomic target sequence in the target cell comprise at least one point mutation. As used herein, the term “point mutation” is intended to mean a genetic mutation where a single nucleotide base is changed, inserted or deleted from a sequence of DNA.

According to the present invention, the CRISPR-Cas system of the invention may be delivered into the target cell, for example, by means of appropriate viral systems. The first and second viral expression vectors of the invention may be packaged into two distinctive separate viral particles.

Thus, in one aspect, the present invention provides a set of two viral particles, wherein one viral particle comprises the first viral expression vector as above defined, and the other viral particle comprises the second viral expression vector as above defined.

The viral particle may suitably be, for example, a lentivirus, a retrovirus, a parvovirus, an adenovirus or an adeno-associated virus (AAV).

Preferably, the viral particle according to the invention is AAV1, AAV2, AAV4, AAV5, AAV6, AAV7, AAV8 or AAV9, more preferably AAV2 or AAV9.

A further aspect of the present invention is an in vitro method for editing a mutant genomic target sequence in a target cell as defined in the appended claim 6. The method of the present invention involves transducing the target cell with a CRISPR-Cas system as above defined or a set of two viral particles as above defined, and culturing the transduced cell under cell culture conditions suitable for the expression of the gRNA and the endonuclease enzyme. The selection of the most appropriate cell culture conditions such as e.g. the medium, the temperature and the incubation time, is well within the reach of those skilled in the art.

In the method of the invention, the intracellular expression of the first and second viral expression vectors leads to the formation of a CRISPR-Cas complex comprising the endonuclease enzyme bound to the gRNA molecule. As illustrated above and depicted in FIG. 1, the mutation-specific gRNA of the CRISPR-Cas complex of the invention drives the endonuclease towards the mutant genomic target sequence based on a complementary-based pairing rule, resulting in the cleavage of said sequence. The double-strand break (DSB) at the genomic target site is repaired by using as template the donor nucleotide sequence consisting of the wild-type sequence of the mutant genomic target sequence in order to stably correct at least one of the mutations in the target sequence and restore the wild-type sequence. In the cell, sequence replacement may be effected, for example, by the homology-directed repair (HDR) correction machinery. Of note, the method of the invention ensures that the replacement of at least one mutation in the mutant target sequence with the wild-type sequence takes place at the naturally occurring site on the genome, thereby enabling the replaced sequence to maintain its native regulation in the target cell. Indeed, the gRNAs used in the present invention are designed to target only a short sequence on the mutated allele, preferably a sequence of 15 to 25 nucleotides in length, which is necessary for the creation of the DSB only in the gene locus of interest, without affecting the remaining part of the gene sequence. Therefore, according to the present invention, the use of the CRISPR-Cas system does not lead to the removal of the entire mutated gene/allele with the reintroduction of the natural wild type copy, but enables to obtain only a DSB at the site of the mutation in order to use the donor DNA as a template for the new synthesis of a small portion of wild-type DNA.

While reference is made to virus mediated delivery, it is understood that other methods of cell transduction may be used within the scope of this invention, as are known in the art. Such methods include, but are not limited to, electroporation, cationic liposome formulations, and calcium phosphate transfection.

As previously illustrated, a target sequence for the CRISPR-Cas system of the invention is present also on the second viral expression vector, either in one copy or in two copies. The gRNA-directed endonuclease cleavage at the one or two copies of the target sequence results in the disruption of the structure of the endonuclease coding unit and, therefore, in the suppression of the transcription and expression of this coding sequence.

In this connection, it should be understood that endonuclease coding sequence auto-cleaving takes place gradually over time so that the CRISPR-Cas system of the invention is able to complete gene editing-mediated correction of the mutant target sequence on the genome in the target cell.

Thus, the method of the invention provides a unique and simple way of performing accurate and efficient editing of a mutant genomic sequence using a CRISPR-Cas system while avoiding at the same time the danger of undesirable off-target effects without the need for any additional system component.

Another aspect of the present invention is an isolated target cell which comprises the CRISPR-Cas system as above defined.

Without limitation, the isolated target cell may be a eukaryotic cell, preferably a mammal cell, more preferably a human cell, even more preferably a human cell affected by a genetic disease.

In one embodiment, the human isolated target cell of the invention is a postmitotic cell or a cell in a post-replicative stage of the cell cycle.

In the context of the present description, the term “post-mitotic” means a terminally differentiated cell that is no longer able to undergo mitosis whereas the term “post-replicative stage” means a cell in the S or G2 phase of the cell cycle.

As previously illustrated, the CRISPR-Cas system of the invention is advantageously characterized by remarkable accuracy and by the absence or significantly reduced occurrence of off-target effects. These features make the CRISPR-Cas system of the invention particularly suitable for use in therapy.

Thus, a yet further aspect of the invention is the CRISPR-Cas system as above defined or the set of two viral particles as above defined, for use as a medicament.

Preferably, the CRISPR-Cas system or the set of two particles according to the invention is suitable for the therapeutic treatment of a genetic disease, more preferably a monogenic disease, even more preferably a monogenic disease selected from the group consisting of Alport syndrome, Pompe disease, Rett syndrome and related disorders, Parkinson's disease, genetic glomerulopathies, lysosomal disorders and monogenic neurodegenerative disorders.

As used herein, the term “genetic disease” refers to any disease, disorder, or condition associated with an insertion, change or deletion in the nucleotide sequence of a genomic locus. Such diseases may include inherited and/or non-inherited genetic disorders, as well as diseases and conditions which may not manifest physical symptoms during infancy or childhood.

Among genetic diseases, the term “monogenic disease” is intended to mean a disease caused by single-gene mutations. It is to be understood that in the present context the term “Parkinson's disease” refers to the monogenic forms of this disorder.

As illustrated in the Experimental section below, the in vivo studies carried out by the present inventors revealed that the present invention makes available a CRISPR-Cas system which is particularly efficient in gene therapy of serious genetic disorders such as Alport syndrome, Pompe disease, Rett syndrome and Parkinson's disease, wherein the affected organs are located in anatomical niches that are difficult to reach by other current therapies. Indeed, the aforementioned pathological conditions affect the function of non-dividing cells with poor regeneration capacity in the kidney, muscle and particularly the CNS, such as terminally differentiated neurons, muscle cells or renal cells.

The present invention also provides a pharmaceutical composition comprising the CRISPR-Cas system as above defined or the set of two viral particle as above defined, in combination with at least one pharmaceutically acceptable vehicle, excipient and/or diluent.

The pharmaceutical composition for use according to the invention is suitable to be administered as a medicament, preferably as a therapy against a genetic disease, more preferably against a monogenic disease, to any mammals, including human beings.

The term “pharmaceutically acceptable” refers to compounds and compositions which may be administered to mammals without undue toxicity at concentrations consistent with effective activity of the active ingredient. Suitable pharmaceutically acceptable carriers (excipients) can be, for example, fillers, disintegrants, glidants and lubricants. Suitable diluents include but are not limited to sterile water, saline buffers, including Phosphate Buffered Saline (PBS) with 5% Sorbitol or glycerol, fixed oils such as synthetic mono- or di-glycerides, polyethylene glycols, propylene glycols and glycerin.

The selection of vehicles, excipients and/or diluents suitable for the pharmaceutical composition of the invention can be determined by a person of ordinary skill in the art by using his/her normal knowledge.

The pharmaceutical composition for use according to the invention is suitable for administration via any of the known administration routes, e.g. topical, oral, enteral, parenteral, intrathecal, epidural and intranasal.

As used herein, parenteral administration includes, but is not limited to, administration of a pharmaceutical composition by subcutaneous and intramuscular injection, implantation of sustained release depots, intravenous injection administration.

A therapeutically effective amount of the pharmaceutical composition of the invention is administered, i.e. an amount capable of producing in the patient the desired effect. Of course, the effective amount may be determined according to various factors, such as for example the type and severity of the disease to be treated, the age, and weight of the patient to be treated, the route of administration, as well as the required regimen.

The pharmaceutical composition for use according to the invention is administered as a single dose or as multiple doses, and as frequently as necessary and for as long of a time as necessary in order to achieve the desired therapeutic effect.

One of ordinary skill in the art can readily determine a suitable course of treatment utilizing the pharmaceutical compositions for use according to the invention.

### BRIEF DESCRIPTION OF THE FIGURES

The following experimental section is provided purely by way of illustration and is not intended to limit the scope of the invention as defined in the appended claims. In the following experimental section reference is made to the appended drawings, wherein:

FIG. 1 is a schematic overview of the gene editing mechanism of the CRISPR-Cas system of the invention, which makes use of two viral expression vectors to deliver the CRISPR-Cas correction machinery into a target cell. A) Schematic representation of the first viral expression vector (“Donor vector”) and the second viral expression vector (“Cas9 vector”) illustrating relevant components in each vector. B) Mutation-specific viral expression vectors are encapsidated into viral particles, preferably adeno-associated viruses (AAVs). Target cells are infected with the viral particles and the viral expression vectors are expressed inside the infected cells. The mutation-specific sgRNA drives the Cas9 enzyme towards the genomic target sequence where a double-strand break (DSB) is introduced by the endonuclease. DBS is repaired by the correction machinery of the cell, which makes use of the Donor DNA as template to restore the wild type sequence by Homology Directed Repair. In addition to mutant target DNA, Cas9 protein is addressed to self-cleaving targets, (sgRNA+PAM sequences; shown as black rectangles) on the “Cas9 vector” itself, resulting in vector cut and shutting-off of Cas9 expression.

FIG. 2 is a schematic representation of the strategy employed to validate viral vectors expression after cell transduction. A) Viral transduction of cells with a Donor vector as shown in FIG. 1, comprising a mCherry/GFP reporter system. In infected cells, mCherry is expressed constitutively and the cell acquires fluorescence (red fluorescence). The GFP protein is not expressed because the sgRNA+PAM sequence located upstream of its coding sequence (indicated as a black rectangle) makes the latter out of frame. B) Coinfection of cells with the Donor vector and a Cas9 vector as shown in FIG. 1. When the Cas9 is expressed, it introduces a break at the sgRNA+PAM sequence between mCherry and the GFP coding sequences, restoring GFP reading frame. Consequently, the cell acquires also green fluorescence.

FIG. 3 shows the results of FACS analysis conducted on different cell types upon infection with AAV2-GFP or AAV9-GFP control viruses. Cells, from left to right: Induced Pluripotent Stem cells (iPSC), neuronal progenitor cells, fibroblasts, iPSC-derived neurons. For each viral serotype, the graphs show the percentage of infected cells which are GFP positive. In each graph, the y axis shows SSC (side scatter), and the x axis shows fluorescence intensity. The dashed rectangle represents the gating for positive cell and the percentage of positive cells is indicated for each condition.

FIG. 4 shows the results of FACS analysis conducted on podocyte-lineage cells harboring mutation in COL4A5 gene upon infection with AAV2-GFP or AAV9-GFP control viruses. The analysis demonstrates a higher infection efficacy of AAV2 compared to AAV9.

FIG. 5 shows the results of in vitro validation of viral expression vectors. Primary fibroblasts were transfected with recombinant plasmids for the c.688C>T-p.Arg230Cys FOXG1 mutation and the c.473C>T-p.Thr158Met MECP2 mutation. a) Representative micrographs showing fluorescence in fibroblasts transfected with the FOXG1 constructs 24 hours post-transfection (40× magnification). b) Graphs showing the results of FACS analysis conducted 48 hours post-transfection on fibroblasts transfected with the FOXG1 constructs or transfected with the reporter plasmid alone. The latter are only mCherry+. In each graph, the y axis shows SSC (side scatter), and the x axis shows fluorescence intensity (FL2-A=mCherry; FL1-A=GFP). For each condition, it is indicated the percentage of positive cells.

FIG. 6 shows that personalized work-flow is necessary to accomplish the best results in editing a specific gene/mutation. In some cases (number 1) a suitable combination of a specific sgRNA, PAM and Cas9 is sufficient to obtain high HDR level, low NHEJ level and low off target level. Other cases may require the selection of different sgRNA and PAMs (number 2). Still other cases may require the replacement of Cas9 with SaCas9 (number 3) or with other high fidelity Cas species (number 4).

FIG. 7 shows suitable viral delivery systems and routes of administration of the CRISPR-Cas system of the invention selected for specific monogenic disorders using disease mouse models. Alport syndrome and related disorders, AAV2 vector, injection into renal artery; Rett syndrome and Parkinson's disease, AAV9 vector, tail vein injection; Pompe disease, AAV9 vector, intramuscular injection.

FIG. 8 shows the results of patient longitudinal analysis conducted on specific cohort of patients in order to identify the most appropriate time window for therapeutic treatment with the CRISPR-Cas system of the invention.

1. Material and Methods

### Example 1.1: Skin Biopsy and iPSCs Reprogramming

Following informed consent signature from patients, skin biopsies (about 3-4 mm³) were performed using the Punch Biopsy procedure on patients with Rett syndrome, Alport syndrome, Parkinson's disease and Pompe disease carrying one or more mutations in the following genes: MECP2, FOXG1 and CDKL5 (Rett syndrome); COL4A3, COL4A4 and COL4A5 (Alport syndrome); GAA (Pompe disease); LRRK2 and GBA (Parkinson disease). Fibroblasts isolated from the biopsies were cultured according to standard protocols and routinely passed 1:2 with Trypsin-EDTA. Fibroblasts at passage 2 or 3 were reprogrammed following the protocol by Hotta and colleagues (Hotta A. et al, “Isolation of human iPS cells using EOS lentiviral vectors to select for pluripotency”; (2009) Nat Methods. 6(5):370-6). Briefly, fibroblasts were infected with a lentiviral vector that expresses the reprogramming factors (OCT-4, SOX-2, c-MYC and KLF-4). Seven days after lentivirus infection, fibroblasts were passed onto mitomycin-C-inactivated mouse embryo fibroblasts (feeders). Emerging iPSCs colonies were manually picked and expanded on feeders for some passages. Established clones that maintained a good hESCs-like morphology were moved to feeders-free culture conditions on Matrigel-coated dishes (BD Biosciences, Milano, Italy) in mTeSR1 medium (Stem Cell Technologies, Grenoble, France). From this time point, cells were routinely passed by Dispase (Stem Cell Technologies, Grenoble, France).

### Example 1.2: Neuronal Differentiation of iPSCs

Induced Pluripotent Stem Cells were differentiated into neuronal progenitors (NPCs) and neurons following the protocol routinely used in the laboratory (Patriarchi T. et al, “Imbalance of excitatory/inhibitory synaptic protein expression in iPSC-derived neurons from FOXG1(+/−) patients and in foxg1(+/−) mice”; (2016) Eur J Hum Genet. 24(6):871-80). Briefly, cell colonies were broken down into single cells with Accutase (Merck Millipore®, Burlington, Mass., United States) and transferred into Aggrewell plates (Stem Cell Technologies, Vancouver, Canada) to allow the formation of Embryoid Bodies (EB)-like structures in NB medium (DMEM:F12 with Glutamax supplemented with 1% N2, 4% B27 without Vitamin A, 55 μM beta-mercaptoethanol and 1% penicillin/streptomycin), containing 200 ng/ml Noggin (R&D System, Minneapolis, Minn., United States), for two days. The cell aggregates were then grown in suspension for two additional days in the same medium and then transferred onto matrigel-coated plates in the same medium for the formation of neuronal rosettes, consisting of neuroepithelial cells arranged in a tubular structure. Twenty-four hours later, the medium was changed to NB supplemented with 200 ng/ml Noggin, 200 ng/ml rh-DKK1 (R&D System, Minneapolis, Minn., United States) and 20 ng/ml FGF2 (Thermo Fisher Scientific Waltham, Mass., United states). Rosettes were manually picked, dissociated and expanded in NB medium with the addition of 10 ng/ml FGF2 and EGF (Thermo Fisher Scientific Waltham, Mass., United states). For subsequent neuronal differentiation, NPCs were plated onto Laminin/Poly-L-Ornithine plates in Terminal differentiation medium (TD)(Neurobasal medium supplemented with 1% N2, 2% B27 with Vitamin A, 15 mM HEPES pH7.4, 1× L-Glutamine, 1× NEAA (Non-Essential AminoAcids), 55 μM beta-mercaptoethanol, 1% penicillin/streptomycin, 200 nM ascorbic acid, 10 ng/ml BDNF, 10 ng/ml GDNF, 10 mM dibutyryl-cAMP) for 30 days. Despite the presence of Noggin and DKK that should direct differentiation toward a forebrain fate, there were obtained heterogeneous cultures containing a variable percentage of non-neuronal cells. Neuronal progenitors and neuronal cells were isolated for quantitative analyses by immunomagnetic cell sorting with magnetic beads-conjugated antibodies against PSA-NCAM and CD24, respectively (Miltenyi Biotec, Calderara di Reno, Bologna, Italy). To enrich cell cultures in post-mitotic neurons, cells at day 15 of neuronal differentiation were treated with Mitomycin C from Streptomyces caespitosus (2.5 μg/ml) (Sigma-Aldrich, Merck, Darmstadt, Germania) which induced apoptosis in proliferating cells but did not affect post-mitotic ones.

### Example 1.3: Muscular Differentiation of iPSCs

To induce muscular differentiation in Induced Pluripotent Stem Cells, the iPSCs were cultured in mTeSR1 medium (Stem Cell Technologies, Grenoble, France), on Matrigel-coated plates, with daily medium changes, until confluent (˜2 days). Then, differentiation into mesodermal-lineage cells was initiated on Day 0 by culturing the cells in the presence of CHIR99021 (5 μM) and BMP-4 (10 ng/ml) in RPMI 1640 medium with 2% B27. Differentiation into Synthetic smooth muscle cells (SMCs) or Contractile SMCs began on Day 3. Synthetic SMCs were produced by culturing the cells under the following conditions: ng/ml VEGF-A and FGFβ in RPMI 1640 and 2% B27 minus insulin (from Day 3 to Day 7); 25 ng/ml VEGF-A and FGFβ in RPMI 1640 and 2% B27 (from Day 7 to Day 9), 10 ng/ml PDGFβ and 3 ng/ml TGFβ in RPMI 1640 and 2% B27 (Day 10 to Day 14). Contractile SMCs were produced by culturing the cells under the following conditions: 25 ng/ml VEGF-A and FGFβ in RPMI 1640 and 2% B27 minus insulin (Day 3 to Day 7); 5 ng/ml PDGFβ and 2.5 ng/ml TGFβ in RPMI 1640 and 2% B27 (Day 7 to Day 14). The differentiated cells were enriched for SMCs by maintaining these cells in 4 mM lactate RPMI 1640 metabolic medium for 4 to 6 days.

### Example 1.4: Podocyte-Lineage Cells Isolation

Podocyte-lineage cells were isolated from urines by following the two-step process previously established by the present inventors (Daga S. et. al., “Urine-derived podocytes-lineage cells: A promising tool for precision medicine in Alport Syndrome”; (2018) Hum Mutat. 39(2):302-314.). Urine samples were processed within 4 hours from their collection and centrifuged at 400×g for 10 minutes. The cell pellet was washed with 10 ml of Washing Buffer (DPBS supplemented with 100 U/ml of Penicillin, 100 μg/ml of Streptomycin and 500 ng/ml of Amphotericin B), and centrifuged again at 200×g for 10 minutes. After the removal of the supernatant, the pellet was re-suspended in 250 μl of Primary Medium (DMEM/high glucose and Ham's F12 nutrient mix (1:1), supplemented with 10% (vol/vol) FBS, 100 U/ml of Penicillin, 100 μg/ml Streptomycin, the REGM SingleQuot Kit supplements and 2.5 μg/ml Amphotericin B) and plated in gelatin-treated culture dishes. Primary medium was added to the culture for the next three days (i.e., 24 hours, 48 hours, and 72 hours after plating). Approximately 96 hours after plating, one-third of the medium was removed and 1 ml of RE/MC medium was added. To prepare RE/MC, the RE and MC medium are mixed in a 1:1 ratio. The RE medium contains the proper amount of each supplement vial of the REGM BulletKit for the RE cell basal medium contained in the same kit. The MC proliferation medium contains DMEM/high glucose supplemented with 10% (vol/vol) FBS, 1% (vol/vol) GlutaMAX, 1% (vol/vol) NEAA, 100 U/ml penicillin, 100 μg/ml streptomycin, 5 ng/ml bFGF, 5 ng/ml PDGF-AB and 5 ng/ml EGF. The proliferation medium was changed daily, until two groups of small colonies were noticed, a first group of cells with a more regular appearance, smooth-edged contours and cobblestone-like cell morphologies, and a second group more randomly arranged with a higher proliferation rate. Cells were split around 9-12 days after plating.

### Example 1.5: sgRNA Design and Viral Expression Vectors Cloning

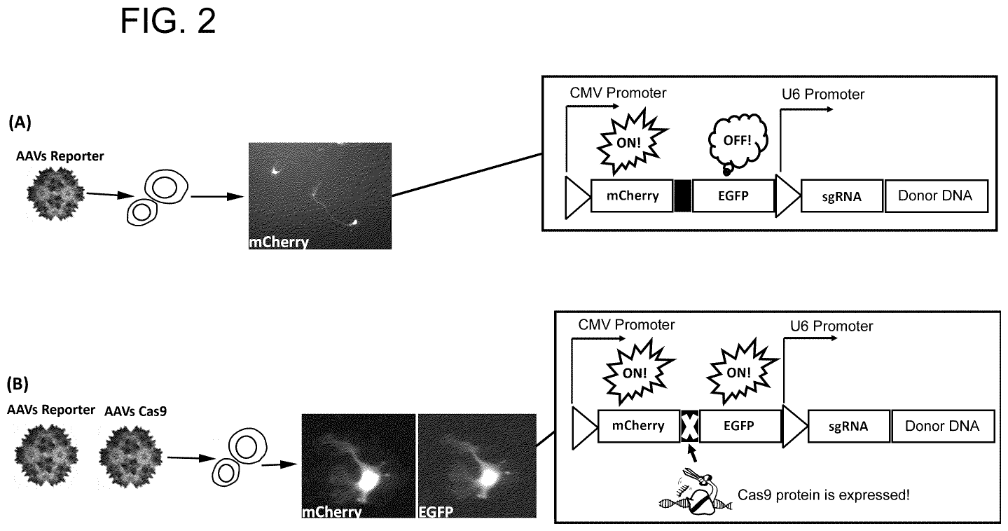

To carry out the in vitro and in vivo experiments illustrated below, the present inventors generated first and second viral expression vectors according to the invention targeting specific disease-associated mutations. The first viral expression vector, referred to also as “Donor vector”, was constructed on the pAAV2.1_CMV_eGFP3 plasmid backbone (Auricchio A. et al., “Exchange of surface proteins impacts on viral vector cellular specificity and transduction characteristics: the retina as a model”; (2001) Hum Mol Genet. 15; 10(26):3075-81). The Donor vector comprises a nucleotide sequence encoding the mutant-specific sgRNA, under the control of the U6 promoter, and a donor nucleotide sequence as above defined which may be used by the correction machinery of the cell as template to replace wild type sequence by Homology Directed Repair. The sgRNAs sequences employed in the experiments, which are complementary to the mutant genomic target sequences of interest, were designed using the MIT CRISPR Design Tool (http://crispr.mit.edu). After the selection of the most suitable sgRNA for each mutant sequence, the corresponding wild-type nucleotide sequence to be used as donor template was designed as a 120 bp sequence centered on the disease-associated mutant nucleotide. The nucleotide sequence encoding the sgRNA and the donor nucleotide sequence were cloned into BbsI and AfIII/AII restriction sites, respectively, in the pAAV2.1_CMV_eGFP3 backbone of the Donor vector. In most experiments, the Donor vector contained also a double mCherry/GFP reporter system, under the regulation of a CMV promoter. The entire coding unit for the reporter system was cloned between the NheI/SpeI unique restriction sites in the pAAV2.1_CMV_eGFP3 vector. As illustrated in FIG. 2, an insert comprising a nucleotide sequence complementary to the mutation-specific sgRNA flanked at its 3′ end by a PAM sequence, was interposed on the vector between the mCherry coding sequence and the GFP coding sequence, rendering the latter out of frame. Because of such configuration, mCherry gene only is expressed constitutively when the vector enters the cell. Upon cell transfection with the vector encoding Cas9, the nuclease is driven by the sgRNA to the sgRNA complementary sequence+PAM sequence located on the Donor vector between the mCherry and GFP genes, and specifically cuts this sequence, reporting the GFP coding sequence in frame, thereby enabling the production of a mCherry/GFP fusion protein. Hence, the two fluorescent proteins of the Reporter system are co-expressed only in the cells where Cas9 is active. Additionally, a WPRE sequence was cloned in the Donor vector, downstream of the reporter system coding sequence, to increase its expression.

For the generation of the second viral expression vector, referred to also as “Cas9 vector”, the present inventors used the PX551 plasmid backbone encoding Streptococcus pyogenes Cas9 (SpCas9) under the control of MECP2 promoter (Swiech L. et al., “In vivo interrogation of gene function in the mammalian brain using CRISPR-Cas9”; (2014) Nat Biotechnol. Oct 19. doi: 10.1038/nbt.3055). In each disease-specific Cas9-vector, a nucleotide sequence consisting of the mutant genomic target sequence (referred to as “target sequence” or “autocleaving sequence”), which is complementary to the sgRNA encoded by the respective Donor vector, along with a PAM sequence located at its 3′ end, were cloned between the MECP2 promoter and the Cas9 coding sequence using unique Agel restriction sites present on the PX551 plasmid. A second autocleaving sequence (sgRNA+PAM) was cloned downstream of the spCas9 cds. The upstream and downstream cuts of the spCas9 cds induce the autocleaving process.

As illustrative non-limiting examples, Table 1 shows the expression vectors used in some embodiments of the CRISPR-Cas system of the invention.

TABLE 1
List of mutation-specific viral expression
vectors of the CRISPR-Cas system

| | | | SEQ ID |
|---|---|---|---|
| Disease | Gene mutation | Vector name | NO. |
| Alport | COL4A5 | pAAV2.1_CMV_eGFP3 | 1 |
| syndrome | c.1871G > A | COL4A5 c.1871G > A | |
| | | (Donor vector) | |
| Alport | COL4A5 | PX551 | 2 |
| syndrome | c.1871G > A | COL4A5 c.1871G > A | |
| | | SpCas9 (Cas9 vector) | |
| Rett syndrome | MECP2 | pAAV2.1_CMV_eGFP3 | 3 |
| | c.473C > T | MECP2 c.473C > T | |
| | | (Donor vector) | |
| Rett syndrome | MECP2 | PX551 | 4 |
| | c.473C > T | MECP2 c.473C > T | |
| | | SpCas9 (Cas9 vector) | |
| Parkinson's | LRRK2 | pAAV2.1_CMV_eGFP3 | 5 |
| disease | c.6055G > A | LRRK2 c.6055G > A | |
| | | (Donor vector) | |
| Parkinson's | LRRK2 | PX551 | 6 |
| disease | c.6055G > A | LRRK2 c.6055G > A | |
| | | SaCas9 (Cas9 vector) | |

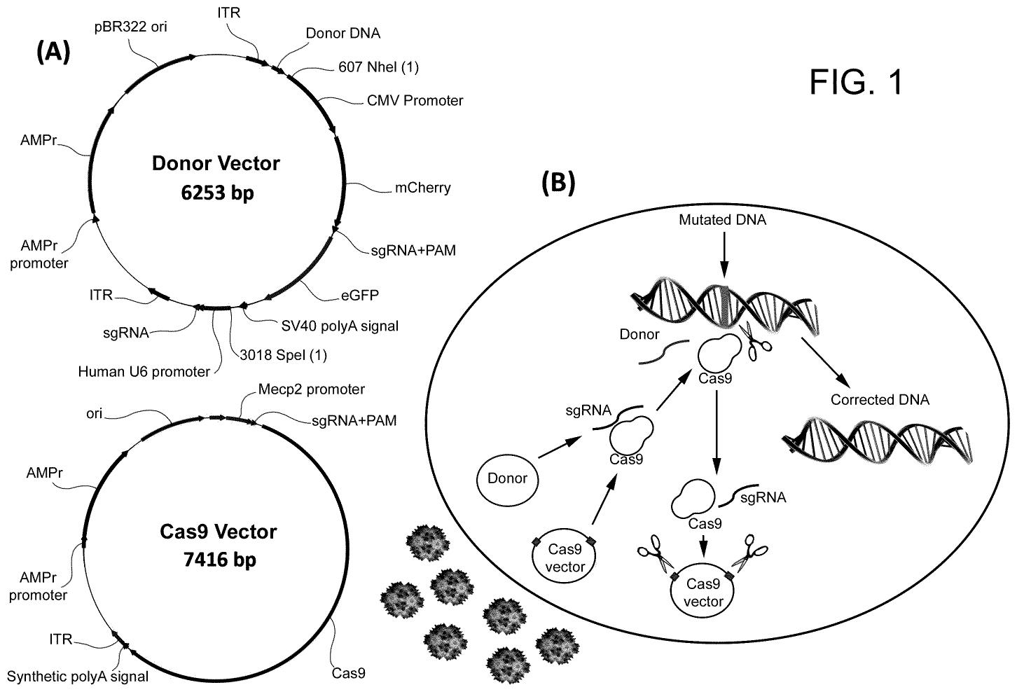

The general structure of the pAAV2.1_CMV_eGFP3 vectors listed in Table 1 is shown in FIG. 1 and comprises the following elements in the 5′ to 3′ direction: donor nucleotide sequence (SEQ ID NO. 7, 8, or 9; see Table 2 below); CMV promoter (SEQ ID NO. 10); mCherry coding sequence (cds)(SEQ ID NO. 11); target nucleotide sequence (SEQ ID NO. 12, 13, or 14; see Table 2 below); 5′-GGG-3′ or 5′-TGG-3′ PAM sequence; GFP cds (SEQ ID NO. 15); U6 promoter (SEQ ID NO. 16); mutant-specific sgRNA coding sequence (SEQ ID NO. 17, 18, or 19; see Table 2 below), and WPRE sequence (SEQ ID NO. 20).

The general structure of the PX551 vectors listed in Table 1 is shown in FIG. 1 and comprises the following elements in the 5′ to 3′ direction: MECP2 promoter (SEQ ID NO. 21); target nucleotide sequence which mediates the endonuclease autocleaving (SEQ ID NO. 12, 13, or 14; see Table 2 below); 5′-GGG-3′ or 5′-TGG-3′ PAM sequence, and Cas9-encoding sequence (SEQ ID NO. 22 for SpCas9 or SEQ ID NO. 23 for SaCas9).

TABLE 2
List of mutation-specific donor sequences,
target sequences and sgRNA coding sequences

| | | | | sgRNA |
|---|---|---|---|---|
| | | Donor | Target | coding |
| | | sequence | sequence | sequence |
| | Gene | SEQ ID | SEQ ID | SEQ ID |
| Disease | mutation | NO. | NO. | NO. |
| Alport | COL4A5 | 7 | 12 | 17 |
| syndrome | c.1871G > A | | | |
| Rett syndrome | MECP2 | 8 | 13 | 18 |
| | c.473C > T | | | |
| Parkinson's | LRRK2 | 9 | 14 | 19 |
| disease | c.6055G > A | | | |

The sgRNA coding sequence of SEQ ID NO. 17 consists of nucleotide sequences SEQ ID NO. 24 (guide-encoding nucleotide sequence) and SEQ ID NO. 25 (scaffold-encoding nucleotide sequence); the sgRNA coding sequence of SEQ ID NO. 18 consists of nucleotide sequences SEQ ID NO. 26 (guide-encoding nucleotide sequence) and SEQ ID NO. 27 (scaffold-encoding nucleotide sequence); the sgRNA coding sequence of SEQ ID NO. 19 consists of nucleotide sequences SEQ ID NO. 28 (guide-encoding nucleotide sequence) and SEQ ID NO. 29 (scaffold-encoding nucleotide sequence).

The first and second viral expression vectors thus generated were transformed in DH5-μ Competent Escherichia coli cells and grown in Luria-Bentani medium; vectors were purified using EndoFree Plasmid Maxi Kits (Qiagen). All constructs were verified by Sanger sequencing (GA3130 Genetic Analyzer, Thermo Fisher Scientific).

### Example 1.6: Cells Electroporation Using Neon Transfection System

In the following, there are illustrated the general and cell type-specific protocols which were set up by the present inventors to achieve cell transfection.

General protocol: cells were passed one-two days prior to electroporation, in order to have cultures 70-90% confluent on the day of the experiment. Electroporation was performed using the Neon® Transfection System following manufacturer protocol (Thermo Fisher Scientific®, Waltham, Mass., United States). Briefly, cells were harvested in growth medium without antibiotics, counted to determine cell density and centrifuged 100-400×g for 5 minutes at room temperature. Cells were subsequently washed with 1× PBS, resuspended in Resuspension Buffer R (Thermo Fisher Scientific) at a final density of 1.0×10⁷ cells/ml and gently pipetted to obtain a single cell suspension. Culture plates were prepared by filling the wells with culture medium containing serum and supplements without antibiotics and pre-incubated in a humidified 37° C./5% CO₂ incubator. A Neon® Tube with 3 ml Electrolytic Buffer (Buffer E for 10 μl Neon® Tip and Buffer E2 for 100 μl Neon® Tip) (Thermo Fisher Scientific) was set up into the Neon® Pipette Station and the desired pulse conditions (specific for each cell type) were set up on the device. The appropriate amount of plasmid was then transferred into a sterile, 1.5 ml microcentrifuge tube and cells were added to the tube. Finally, the cells/plasmid mixture was transferred into a Neon® Tip that was inserted into the Neon® Pipette for electroporation.

Fibroblasts transfection: Primary fibroblasts were harvested with Trypsin Solution (Irvine scientific Santa Ana, Calif., United States) and counted. A total of 4×10⁵ cells were transfected using Neon® Transfection System with 10 ug of DNA (5 μg of the first viral expression vector containing also the Reporter system—Donor vector —, and 5 μg of the second viral expression vector containing the Cas9 coding sequence—Cas9 vector) and the following instrument parameters: 1700 Pulse Voltage (V), 20 Pulse Width (ms) and 1 Pulse number. Untreated cells were used as negative control, while cells electroporated with a viral expression vector encoding only EGFP were used as positive control of transfection. After treatment, cells were plated in 60 mm plates with medium without antibiotics. Transfection efficiency was established with fluorescence microscopy and FACS (Fluorescence Activated Cell Sorting) analysis 48 hours post-transfection.

Podocyte-lineage cells transfection: a total of 2×10⁵ of urine-derived podocytes isolated from affected patients harboring mutations in COL4A3 gene [(c.1567G>A p.(Gly856Glu))] and COL4A5 gene [(c.1871G>A p.(Gly624Asp))] were electroporated using the Neon® Transfection System in accordance with manufacturer's protocol. A total of 5 μg of the first viral expression vector containing also the Reporter system (Donor vector) and 15 μg of the second viral expression vector containing the Cas9 coding sequence (Cas9 vector) in a 1:3 ratio, were co-transfected using the following instrument settings: 1.150 Pulse Voltage [V], Pulse Width [ms], 2 Pulse Number. Untreated cells were used as negative control while a plasmid encoding only EGFP was used as positive control for transfection efficiency. After transfection, cells were plated on 6 well plates coated with 0.1% human gelatin (Merck Millipore®, Burlington, Mass., United States), using RE/MC growth medium without Penicillin/Streptomycin. Transfection efficiency was confirmed with fluorescence microscopy and FACS analysis 48 hours post-transfection.

iPSCs cells transfection: cells were harvested with Accutase (Stem Cell Technologies) and 4×10⁵ cells were transfected using Neon® Transfection System with 10 μg of DNA (5 μg of the Donor vector containing also the Reporter system and 5 μg of the Cas9 vector) using the following instrument parameters: 1100 Pulse Voltage (V), 30 Pulse Width (ms) and 1 Pulse number. Untreated cells were used as negative control, while cells electroporated with a plasmid encoding only EGFP were used as positive control of transfection. Following treatment, cells were plated on 12 well plates with medium without antibiotics. Transfection efficiency was confirmed with fluorescence microscopy and FACS analysis 48 hours post-treatment.

Neuronal Precursors cells transfection: neuronal precursors (NPCs) were transfected with a mutation-specific viral expression vector to determine transfection efficiency and Cas9 transduction. Cells were detached from plate with Accutase (Stem Cell Technologies), diluted 1:1 with Dulbecco's modified Eagle Medium (GIBCO, Thermo Fisher scientific, Waltham, Mass., United States) and transfection was performed using Neon® Transfection System according to manufacturer's protocol. A total of 4×10⁵ cells and 10 μg of DNA (5 μg of the Donor vector containing also the Reporter system and 5 μg of the Cas9 vector) were used and the instrument was set up with the following parameters: 1300 Pulse Voltage (V), 20 Pulse Width (ms) and 1 Pulse number. After treatment, cells were plated on 12 well plates coated with poly-L-Ornithine and Laminin (Sigma-Aldrich, Merck Millipore®, Burlington, Mass., United States) with medium without antibiotics. Transfection efficiency was confirmed with fluorescence microscopy and FACS analysis 48 hours post-treatment.

### Example 1.7: Neurons Transfection Using Lipofectamine

A total of 1×10⁵ neural precursor cells (NPCs) were plated on 12 well plate coated with poly-L-Ornithine and laminin (Sigma-Aldrich, Merck Millipore®, Burlington, Mass., United States) allowed to attach in NB medium (DMEM:F12 without Glutamax supplemented with 1% N2, 4% B27 with Vitamin A, 55 μM beta-mercaptoethanol and 1% penicillin/streptomycin) for 3-4 hours and then the medium was changed to TD medium (Neurobasal medium supplemented with 1% N2, 2% B27 with Vitamin A, 15 mM HEPES pH7.4, 1× L-Glutamine, 1× NEAA (Non-Essential AminoAcids), 55 μm beta-mercaptoethanol, 1% penicillin/streptomycin, 200 nM ascorbic acid, 10 ng/ml BDNF, 10 ng/ml GDNF, 10 mM dibutyryl-cAMP). After 2 days cells were treated with Mitomycin C from Streptomyces caespitosus (Sigma Aldrich, Saint Louis, Mo., United States) to positively select post-mitotic neurons. Neurons were transfected with mutation-specific viral expression vectors on day fifteen. Transfection was performed using Lipofectamine 2000 (Invitrogen Corporation, Carlsbad, Calif., United States) in accordance with manufacturer's protocol. Transfection efficiency was confirmed with fluorescence microscopy and FACS analysis during the period from 3 to 8 days post-treatment.

### Example 1.8: AAV2/AAV9 Infection

The viral expression vectors according to the invention were encapsidated at the Vector Core Facility at TIGEM (http://www.tigem.it/core-facilities/vector-core) to produce Adeno-Associated Viral preparations. AAV serotypes 2 and 9 were produced and tested on all relevant cell types.

AAV9 infection was preceded by Neuraminidase treatment in order to expose N-Linked-Galactose that acts as AAV9 receptor. To this aim, the cells were treated with 50 mU of Endo-α-Sialidase (Neuraminidase) for 2 hours at 37° C. The medium containing Neuraminidase was then removed and fresh medium, containing AAV9 and without FBS and antibiotics, was added. The plate was centrifuged for 1 minute at 1100 rpm and then incubated for 1 hour and 30 minutes at 4° C. Subsequently, fresh medium with FBS was added and the plate was incubated overnight at 37° C. After 24 hours the medium containing the virus was removed and fresh medium was added. After 48 hours fluorescence was quantified by FACS.

No pretreatment was required for AAV2 infection because the receptor, membrane-associated heparan sulfate proteoglycan, is naturally present unmasked on the cell surface.

### Example 1.9: Flow Cytometry Analysis and Cell Sorting

To carry out flow cytometry analysis, the present inventors employed the flow cytometer BD FACSaria II (BD Biosciences-US), which acquired 10,000 events/well. Data were analyzed using a flow cytometry software package, such as FlowJo v7.5. The gate for the live cells was based on forward (size) and side (granularity) scatters; a second gate was determined for live cells based on high mCherry and EGFP-positivity to determine the percentage of cells which were transfected with both the first (Donor vector) and second (Cas9 vector) viral expression vectors. In order to isolate EGFP-positive cells for subsequent analyses, cells were detached and resuspended in PBS/EDTA 20 mM. Cells were placed on ice and then sorted using BD FACSAria II. The DNA was extracted from EGFP+ cells using the Qiamp DNA micro kit (Qiagen, Hilden, Germany).

### Example 1.10: Ion Torrent S5 Sequencing

The Ion AmpliSeq 2.0™ Library Kit (Life Technologies™, Carlbad, Calif.) was used for library preparation. The kit allowed obtaining a barcoded library of the genes involved in the disease under analysis, according to the Life Technologies manufacter's protocol. Libraries were purified using Agencourt AMPure XP system and quantified using the Qubit® dsDNA HS Assay Kit reagent (Invitrogen Corporation, Life Technologies™), pooled at an equimolar ratio, annealed to carrier spheres (Ion Sphere™ Particles, Life Technologies) and clonally amplified by emulsion PCR (emPCR) using the Ion Chef™ system (Ion Chef™, Life Technologies). Ion 510™, 520™ or 530™ chip were loaded with the spheres carrying single stranded DNA templates and sequenced on the Ion Torrent S5 using the Ion S5™ Sequencing kit, according to the manufacturer protocol of Life Technologies.

### Example 1.11: NGS Analysis

To perform NGS analysis, the FASTQ files for transfected cell samples and the relative controls, which are returned by the sequencing platform for each disease (S5 Torrent Server VM), were uploaded to the online analysis tool CasAnalyzer (http://www.rgenome.net/cas-analyzer/M) along with the sgRNA sequences designed for each mutation, the donor nucleotide sequence and the mutant target sequence. The percentage of achieved homology-directed repair (HDR) was thus obtained, considering a suitable comparison range (R) of nucleotides around the cut site.

The software returns a table used for further bioinformatic analysis by ad hoc scripts (Python™ Software Foundation License) for indels computation.

The .bam and .bai files, for each sample, were uploaded on IGV Visualization Software (Broad Institute, Cambridge, United States) to precisely visualize the percentage of editing for any mutated nucleotide.

### Example 1.12: Off-Targets Analysis with GUIDE-Seq

The present inventors carried out dedicated experiments in order to assess off target activity of the CRISPR-Cas system of the invention. To this aim a Guide-seq analysis was performed as illustrated in Tsai S Q et al, “GUIDE-seq enables genome-wide profiling of off-target cleavage by CRISPR-Cas nucleases”; (2015) Nat Biotechnol. 2015 Feb; 33(2):187-197), using HEK293 cells harboring patient-specific mutations as in vitro model. HEK293 cells were cultured in Advanced DMEM (Life Technologies™, Carlsbad, Calif., United States) supplemented with 10% FBS, 2 mM GlutaMax (Life Technologies™, Carlsbad, Calif., United States) and 1% penicillin/streptomycin at 37° C. with 5% CO₂. Cells were seeded in six well-plate at 2×10⁵ cell/well the day before the experiment. HEK293 cell were transfected with X-tremeGENE™ HP DNA Transfection Reagent according to manufacturer's instructions, using 250 ng of a first plasmid encoding the mutation-specific sgRNA and donor sequences (Donor vector), 750 ng of a second plasmid encoding spCas9 (Cas9 vector), and 10 pmoles of annealed double-stranded oligodeoxynucleotides (dsODN) that are integrated at the sites of breaks generated by Cas9 by means of NHEJ. The dsODN integration allows the subsequent identification of off-target breaks using unbiased amplification and next-generation sequencing (see below). In addition, there were added 50 ng of a PBML4 plasmid which encodes for puromycin resistance and is used to positively select transfected cells. Selection was performed 48 hours post transfection with 1 ug/ml puromycin. Transfection efficiency was also evaluated using FACS analysis.

Genomic DNA (gDNA) was isolated from transfected cells using the Wizard® SV Genomic DNA Purification System (Promega), sheared with a Diagenode Bioruptor instrument to an average length of 500 bp, by using a total of 1 μg of gDNA in 100 μl of DNA water to match the instrument specification. After quantification with Qubit (Invitrogen), the DNA was purified with AMPure XP beads (Beckman Coulter) according to GUIDE-seq protocol. A total of 400 ng of gDNA were used as input for library preparation, followed by end-repaired, A-tailed and ligated to half-functional adapters, incorporating a 8-nt random molecular index. Two rounds of nested anchored PCR, with primers complementary to the oligo tag, were used for target enrichment. Libraries were obtained using Kapa Biosystem Kit and quantitated with Illumina Library Quantification Kit according to manufacturer's instructions. Subsequently libraries were denatured and loaded onto the Illumina Miseq instrument according to Illumina's standard protocol for sequencing with an Illumina Miseq Reagent Kit V2-300 Cycle. Sequencing data were analyzed using free web tools such as Tracking of Indels by Decomposition (TIDE: http://tide.nki.nl) and Interference of CRISPR Edits (ICE: https://ice.synthego.com/#/) to quantitate editing efficacy and to simultaneously identify and quantitate the predominant unwanted insertions and deletions (indels) in the targeted pool of cells.

### Example 1.13: Editing Efficiency

To assess the editing efficiency of the CRISPR-Cas system of the invention, two different approaches were employed as described below.

System 1: Editing efficiency of LRRK2 (p. (Gly2019Ser)) and GBA (p. (Leu444Pro)) CRISPR/Cas9-AAV system in murine cells.

Primary neurons from Parkinson's disease (PD) mouse model were co-transducted with (i) AAV vector comprising the saCas9 coding sequence (SEQ ID NO. 6), (ii) AAV vector comprising a nucleotide sequence encoding a sgRNA targeting the (p.(Gly2019Ser)) mutation ((pAAV2.1_CMV_eGFP3 LRRK2 c.6055G>A of SEQ ID NO. 5), or (iii) AAV control vector comprising a lacZ sgRNA coding sequence. As outcome measures, the reduction of neurites complexity (analysis of number and length of neurites by using the NeuronJ software), and the LRRK2 kinase activity (analysis of LRRK2 kinase phosphorylation and of Rab10 substrate by immunoblotting) were evaluated, compared to wild type (WT) levels. For the CRISPR/Cas9-AAV targeting GBA p.Leu444Pro, GBA enzymatic activity was measured in cell lysates as previously described (Ferrazza R. et al., “LRRK2 deficiency impacts ceramide metabolism in brain”; (2016) Biochem Biophys Res Commun. 23; 478(3):1141-6). The efficacy of gene-corrected cells in rescuing the reported mitochondrial defects was also tested. Sequencing methods to test for off targets events were performed.

System 2: Editing efficiency of COL4 and GAA CRISPR/Cas9-AAV system in podocytes and muscle cells.

To transduce Alport syndrome (ATS) podocytes and muscle cells from Pompe patients, a multiplicity of infection (MOI), between 10⁴-10⁶, were used to assess the specificity of the gene correction editing system for in-vitro infection. ATS podocytes were co-transfected with viral expression vectors pAAV2.1_CMV_eGFP3 COL4A5 c.1871G>A (SEQ ID NO. 1) and PX551 COL4A5 c.1871G>A spCas9 (SEQ ID NO. 2). For assesing transfection efficiency, the present inventors took advantage of the mCherry/Green reporter system, which produces a switch from Red to Green fluorescence easily detectable 72 hours after infection. The unique Green fluorescent cell population was recovered using FACS sorting technology in order to perform Sanger Sequencing and Whole-Exome Sequencing (WES) to evaluate efficiency, correction specificity and off-target index of the CRISPR-cas system of the invention. The primary aim was considered achieved if in-vitro correction was obtained in a range between 40% and 50% of the cell population.

### Example 1.14: Mouse Models

Peripheral AAV Delivery

Mice were treated with different dosages of AAVs harbouring the CRISPR/Cas9 correction vectors, ranging from 5×10¹⁰ to 5×10¹³ at different ages almost corresponding to the therapeutic window assessed in humans for the different diseases treated.

CRISPR/Cas9 AAV particles were administered via injection into the tail vein. Different viral doses (dose-escalating approach) were tested; repeated injections with a defined time schedule were also tested. Young animals (P1-P6) were injected to simulate the conditions for a trial in patients. The efficiency and specificity of gene correction with the different treatment conditions was evaluated in different tissues and brain regions (cortex, hippocampus, striatum, cerebellum). For this purpose, tissues were isolated 60 days following treatment (for repeated injections, the last one is considered), and DNA extracted from these tissues underwent both T7E1 assay and deep sequencing to evaluate the efficiency of correction and the possible generation of indels due to Cas9 cut without HDR-directed correction. Accurate characterization of the general health status of treated mice was performed, including regular monitoring of body weight and standard blood chemistry. Animals were sacrificed 60 days following treatment to perform histological analysis for potential liver toxicity and damage to other organs. Tissues from sacrificed animals were also employed for the characterization of the biodistribution of the viral genome in different tissues by performing quantitative RT-PCR. Effective viral doses as well as injection numbers in reverting mutants phenotype were determined by comparing untreated and treated Knock-in mice. In particular, animals of the following groups were used: i) KI mice injected with LacZ CRISPR/Cas9-AAV, ii) WT mice injected with LacZ CRISPR/Cas9-AAV and iii) KI mice injected with mutation specific-CRISPR/Cas9-AAV.

Mice were also injected intraperitoneally with doses of CRISPR/Cas9-AAV, ranging from 10¹¹ to 10¹⁵ viral genomes (vg)/Kg.

GFP fluorescence emission distribution was visualized in real-time by using IVIS Lumina III system in combination with in vivo two-photon confocal imaging. Animals were imaged at 30, 60, 90 and 120 days post-injection and the optimal dose and timing was identified and used for the following experiments. The percentage of corrected cells was determined by GFP imaging of fixed tissue slices.

With specific regard to Parkinson's disease mouse model, the rescue of LRRK2-associated phenotypes in gene-corrected mice was evaluated based on the reduction of neurites complexity mediated by LRRK2 mutation. The neurites number and length of dopaminergic neurons was analyzed using organotypic slices. Brain slices (300 nm), in the number of four for each hemisphere, which include the nigro-striatal pathway, were fixed and then stained. GFP- and tyrosine hydroxylase (TH)-positive neurons (at least 50) were analyzed as above described. The evaluation carried out by the present inventors was also based on LRRK2 kinase activity restoration. LRRK2 S1292 auto-phosphorylation and LRRK2-mediated Rab10 phosphorylation in FACS sorted GFP-positive cells from midbrain was monitored by immunoblotting (IB). Moreover, the rescue of S129 phosphorylated aSyn accumulation was considered in the present analysis. Phospho aSyn in FACS sorted GFP-positive cells from midbrain was evaluated by D3.

In the present invention, the rescue of GBA-associated phenotypes in gene-corrected animals was evaluated by analyzing GBA activity in brain and peripheral tissues (kidney, lung, liver) lysates from GFP-sorted cells from the animals. Furthermore, aSyn accumulation/phosphorylation was determined by D3 and immunohistochemistry on different brain regions (midbrain, striatum, cortex) of 4-months old (8 weeks post-injection) and 8-months old (6 months post-injection) animals (5 animals per group, per age). Finally, the restoration of mitochondria defects by administration of L444P GBA CRISPR/Cas9-AAVs was evaluated in brain slices of gene-corrected and control mice by transmission electron microscopy (TEM) of fixed tissue.

Intra-Renal Artery AAV Delivery in the Dog Model

In this animal model, ATS affected male dogs were treated at 8 weeks of age, which is the age typically preceding the occurrence of microalbuminuria, currently representing the first clinical indication of ATS disease in dogs. A single femoral artery catheterization was used to deliver either the wt-AAV2-CRISPR/Cas9 based correction system (vectors of SEQ ID NO. 1 and 2) or the mut-AAV2-CRISPR/Cas9 system in-vivo, injecting into both renal arteries under fluoroscopic guidance. This procedure was performed at the following two dosages: 10¹¹-10¹² and 10¹³-10¹⁴. The mut-AAV2-CRISPR/Cas9 was used as a negative control. Dogs were routinely evaluated for 15 months or until euthanasia, if necessary due to end-stage renal disease and/or severe clinical illness. Urinalysis, urine protein-creatinine ratio (UPC), and serum creatinine concentration (sCr) was performed every 2 weeks. A more comprehensive evaluation (complete blood count, chemistry panel, and iohexol clearance) was performed at baseline (8 weeks of age, immediately prior to treatment) and at least every 3-4 months thereafter, generally corresponding to the collection of kidney biopsies. At each time of sampling, at least 5 ml urine and 5 ml blood were collected for immediate testing and for archiving at −80° C. for potential future testing. When iohexol clearance was to be performed, an 8-point sampling protocol was used, with 2 ml blood collected at each sampling time (approximately 16 ml total). Kidney biopsies were collected after the first month and after three, six and twelve months post-infection. Multiple biopsies were collected at each time point for various pathologic evaluations. Body condition score and blood pressure were determined on a monthly basis starting at 8 weeks of age.

Gene therapy efficacy was evaluated according to the following parameters: i) ability to trigger production of a correct α3-α4-α5heterotrimer and consequently a proper GBM production by the podocytes and ii) recovery (or partial recovery) of renal function after correction.

Evidence of proper GBM production in the collected kidney biopsies was assessed by immunostaining for COL4 alpha chains using COL4A3, COL4A4 and COL4A5 antibodies at the selected time points post-infection. As indicated above, renal function was routinely evaluated to monitor for clinical evidence of disease progression. Urine-derived podocytes were evaluated monthly by assessing podocyte function and the rate of podocyte loss. Podocyte loss was determined as ratio between podocytes number in the urine pellet obtained from a specified volume of urine, normalized to urine creatinine concentration and/or microhematuria (personal data). Podocyte function was assessed using albumina intake and VEGF-A production, parameters associated with differentiated and functional podocytes. The therapy was deemed partially effective if the onset of azotemia (sCr>1.2 mg/dL) was delayed beyond 40 weeks of age and/or end-stage disease (sCr>5 mg/dl) was delayed beyond 60 weeks of age, combined with at least partial recovery of COL4α3-α4-α5 production in the absence of relevant side effects. The therapy was deemed substantially effective if azotemia did not develop within 1 year post-treatment and/or if a correction range between 40% and 50% was estimated in urine derived-podocytes over the time and podocyturia and proteinuria were mild to absent.

Intra-Muscular AAV Delivery

Neonatal GAA knockout (KO) mice were bilaterally injected with GAA CRISPR/Cas9 dual vector system into the gastrocnemius muscles at birth and at P1 to maximize the amount of injected vector (7×10¹⁰ vg total in 40 μl, 10 μl per GA at P0 and at P1), or into both the tricipites and gastrocnemius muscles (7×10¹⁰ vg to 1.6×10¹¹ vg total in 80 μl, 10 μl per muscle at P0 and at P1). Injected mice were compared with age-matched heterozygous mice for spontaneous activity at ˜100 days of age using an actimeter system (Activmeter; Bioseb, Vitrolles, France). This apparatus uses vibrations within the cage to measure locomotion and infrared photocell detectors to record the number of rearing movements. The distance covered (cm), the average speed(cm/s), and number of rearing movements were measured over a 60-minutes period. Kaplan-Meier survival curve and weight curve of neonatal GAA knock-out mice injected into gastrocnemius and both gastrocnemius and tricipites were calculated.

### Example 1.15: Human Studies

The present inventors carried out longitudinal analysis of patients with the aim to translate CRISPR/Cas9-AAV approach into a pilot study.

For all the above mentioned disorders (Alport Syndrome, Parkinson Disease, Pompe Disease, Rett Syndrome), the trials were performed to evaluate the safety, tolerability and efficacy of gene therapy in patients with the CRISPR-Cas system of the invention. To set up a tailored design for studies aiming to clinically translate the CRISPR/Cas9-AAV approach, a cohort of patients harbouring disease-associated genomic mutations was assembled to determine clinical features and natural disease progression. A cohort including 190 patients (60 with Rett syndrome, 10 with Pompe disease, 80 with Alport syndrome and 40 with Parkinson's Disease) was enrolled to assess the most appropriate therapeutic window for gene delivery. At the baseline visit there were collected informed consent completed and signed, inclusion and exclusion criteria, medical/surgical history, current medications, smoking status, height and weight. The evaluation of clinical outcome under the standard protocol has been studied for at least 20 years back for Rett syndrome and Pompe Disease and at least for 50 and 80 years back for Alport syndrome and Parkinson's Disease, respectively.

The specific design of the trial in terms of viral doses was defined based on the previously described studies in mice or in dogs. A design involving viral doses between 6.7×10¹³ vg/kg and 2.0×10¹⁴ vg/kg was considered. Planning of the clinical trial included the definition of viral dosages, single versus multiple doses and, in the latter case, interval between doses.

Therapy effects were evaluated over a period of at least twelve months. Patients were tested at baseline and follow up visits were scheduled at days 7, 14, 21, 30 and then once every month until the conclusion of the trial. During the baseline and follow-up visits patients underwent clinical, biochemical and instrumental examination according to the specific disease. A primary analysis for efficacy was assessed when all patients reached 12 months post-dose. A follow-up safety analysis was completed at the time point at which the last patient reached 24 months post-dose. Upon completion of the 2-year study period, patients are monitored annually as per standard of care for up to 15 years.

Patients with Rett syndrome were evaluated when necessary with visits at home. Evaluation included clinical examination, EEG analysis, Quality of Life Assessment (CHQ), the Clinical Severity Scale (CSS) and the Motor Behavioral Assessment (MBA). Baseline GABA receptor activity was evaluated and used both as biomarkers of disease progression and to monitor the efficacy of the treatment. The combined analysis of all the above measurements allowed defining the best timing for treatment initiation. It is also essential to define clinical parameters which have to be considered prior to the treatment and identify the most appropriate tests and outcome measures for the evaluation of treatment efficacy at the end of the trial.

Patients with Parkinson Disease carrying disease-associated mutations were selected and characterized according to dedicated clinical rating scales for motor and non-motor features (eg. MDS-UPDRS, Parkinson's disease questionnaire, Hoehn-Yahr and Non-motor symptom assessment scale and other available validated questionnaires). Clinical assessments were videotaped and performed using a neuropsychological validated PD battery and tests for cardiovascular autonomic function. All patients received a baseline MRI scan and a 123I-ioflupane-dopamine transporter (DAT) scan. Patients with sympathetic dysautonomia also received a 123I-meta-iodobenzylguanidine (MIBG) scan. The patient cohorts are assessed longitudinally at yearly intervals to quantify the disease natural progression using the same assessment parameters. DAT scan and, eventually, the MIBG scan are repeated two years after baseline assessment.

Efficacy was evaluated on the basis of clinical, biochemical and instrumental examination. Additional specific outcome measures were defined on the basis of the results of the tests carried out in mice.

Regarding ATS, at the baseline visit clinical and biochemical parameters (GFR, creatininemia, creatinine clearance microalbuminuria/proteinuria levels) were collected. At each follow-up visit physical exam data were annotated including detailed cardiological exam, body weight, height measurement, ECG, hematology (Hb, Ht, WBC-diff, platelets), biochemistry (ALT, AST, creatinine, creatinine clearance, proteinuria/creatinuria levels), eGFR, urinary pregnancy test (for women of childbearing potential), diet.

Regarding Pompe Disease, at baseline visit, as primary outcome measure, the cardiac function was measured by the left ventricular mass Z-score (LVM-Z). Z-Scores indicate the number of standard deviations (SD) from the mean in a normal distribution. A negative change from baseline indicates a decrease and positive change from baseline indicates an increase in LVM Z-score. The normal range is −2 to 2 and greater than 2 may indicate left ventricular hypertrophy.

Motor development status was also assessed by the Gross Motor Function Measure-88 Scale (GMFM-88) total percent scores and repeated measures were performed at the follow-up visits. GMFM-88 is an 88-item measure to detect gross motor function. In alternative, for early onset disease the Hammersmith modified scale was used to test motor function.

2. Results

In order to demonstrate the efficacy, safety and therapeutic potential of the CRISPR-Cas system of the invention, the present inventors carried out dedicated proof-of-principle studies using as disease model four different monogenic disorders, namely Alport syndrome, Rett syndrome, Pompe disease and Parkinson's disease.

### Example 2.1: AAV2 and AAV9 Provide the Highest Infection Efficiency

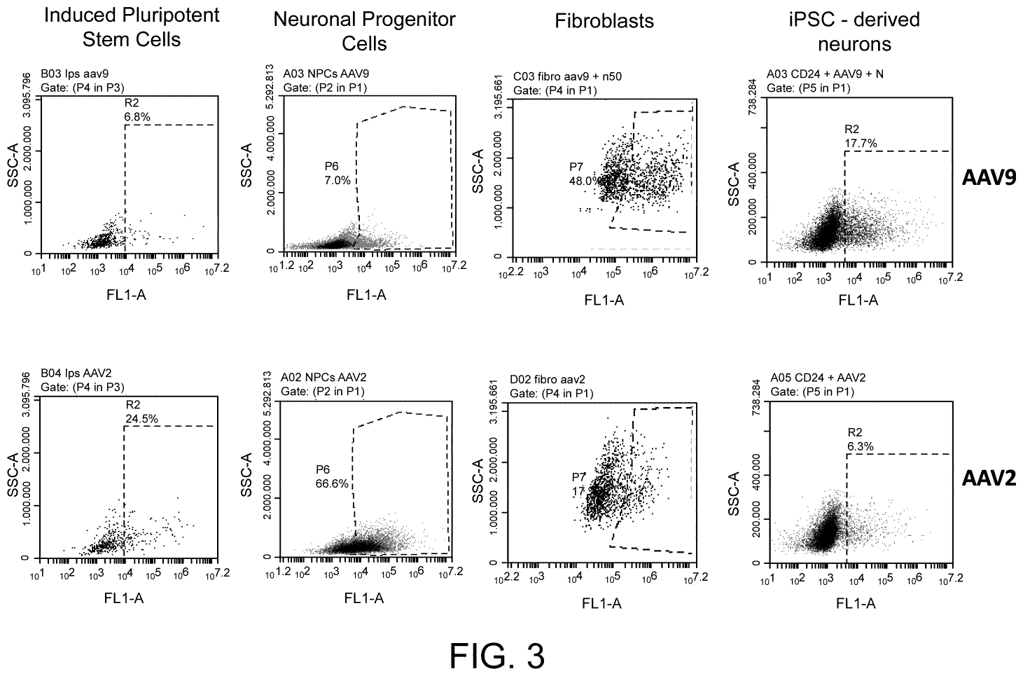

To set up an efficient infection protocol suitable for various patient-derived cell types, different cells were infected with control viruses expressing only the EGFP protein (pAAV2.1_CMV_eGFP3 viral expression vector). In such experiments, both the AAV2 and AAV9 serotypes were tested. As shown in FIG. 3, when using AAV2, a 24% infection level was reached in induced Pluripotent Stem cells and a 67% infection level was reached in neuronal precursors with a multiplicity of infection (MOI) of 1×10⁵ and 2×10⁵, respectively. When using AAV9, an infection level of 48% was determined in fibroblasts and of 17% in neurons with a multiplicity of infection (MOI) of 3×10⁵ (FIG. 3).

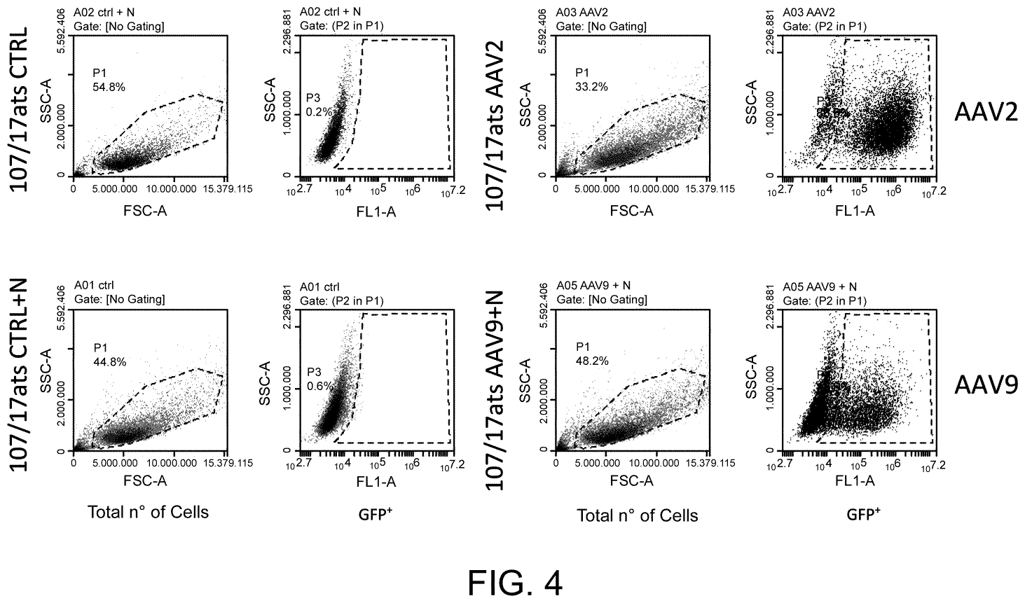

Since a specific kidney tropism has been reported for both AAV2 and AAV9, the present inventors tested both serotypes on podocytes-lineage cells, to define the most suitable serotype to be used in order to achieve the highest transfection efficiency. Using AAV2 virus and a multiplicity of infection (MOI) of 10⁵, an infection efficiency of 86% was reached in podocyte-lineage cells compared to an infection level of only 40% obtained using an AAV9 vector (FIG. 4).

### Example 2.2: Correction of Mutant FOXG1 and MECP2 Genes in Cell Systems

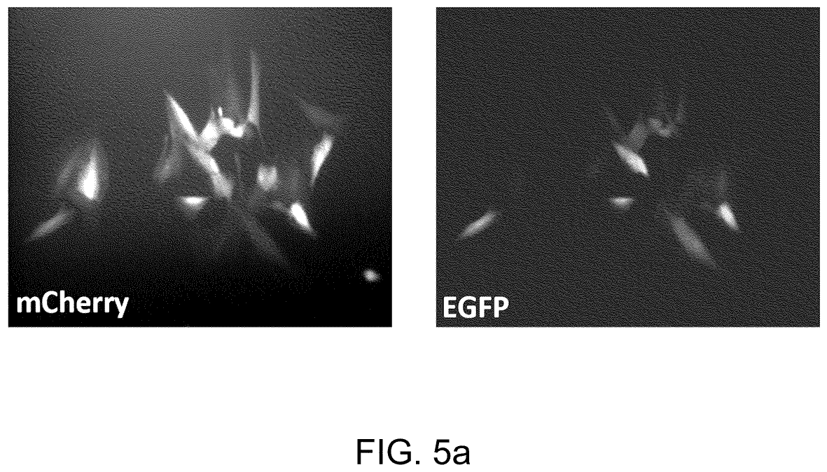

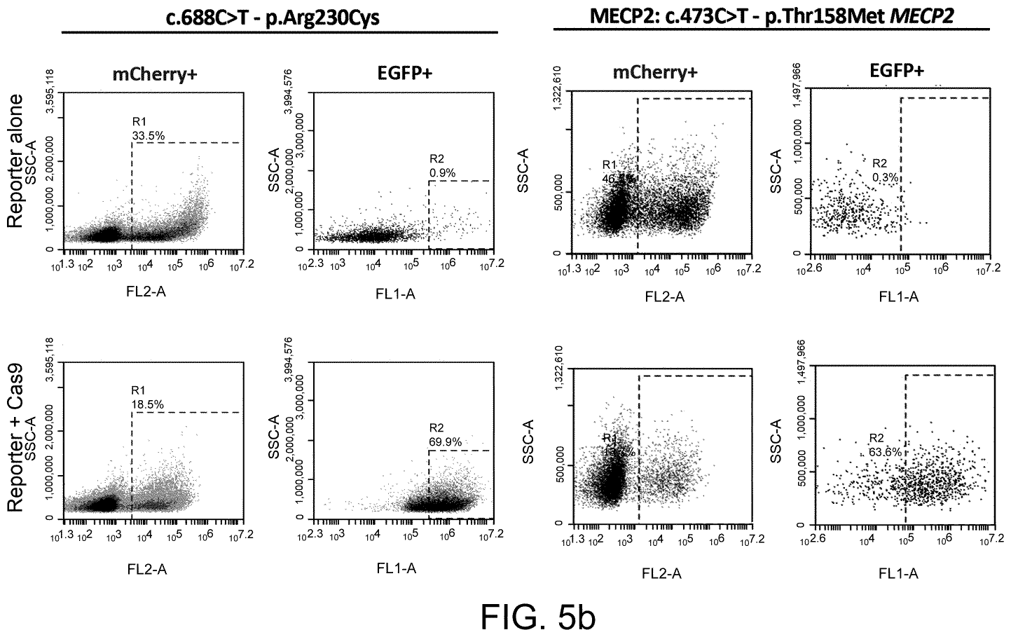

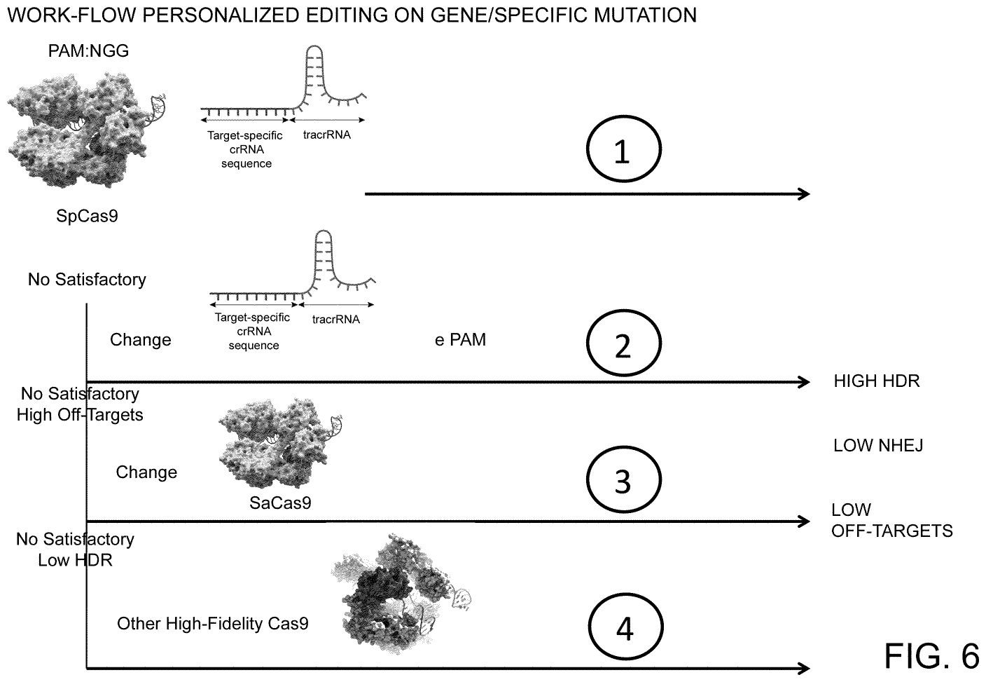

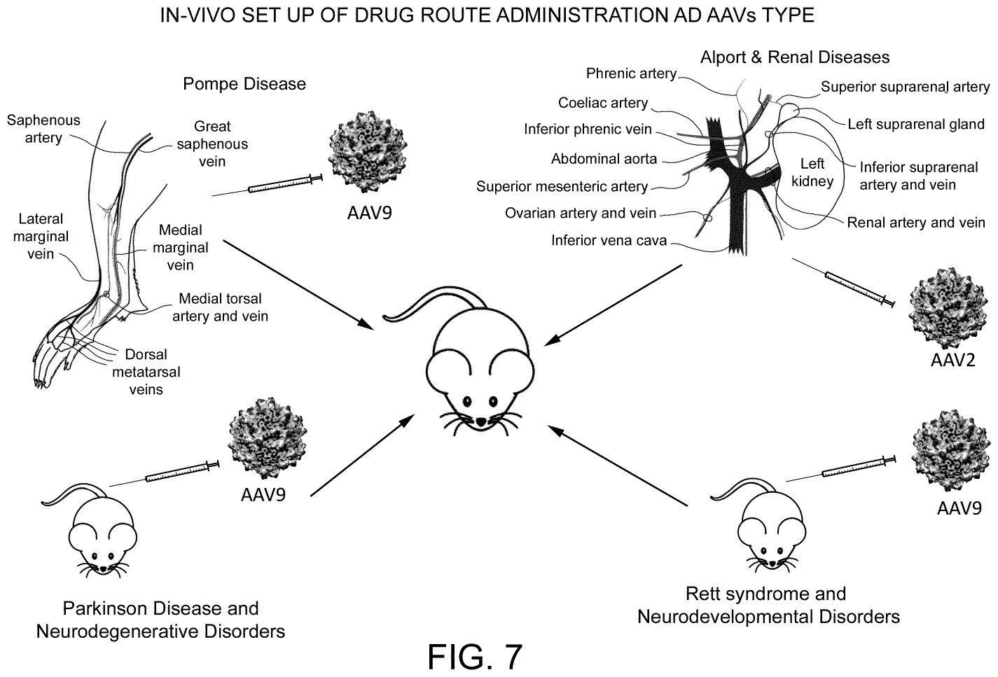

To assess gene editing efficiency of the CRISPR-Cas system of the invention, patients with Rett Syndrome were selected harboring mutations in MECP2 [(c.473C>T (p.(Thr158Met))] or FOXG1 [c.688C>T (p.(Arg230Cys)) or c.765G>A (p.(Trp255*))] genes. Viral expression vectors specific for each mutation, i.e. comprising a mutation-specific gRNA coding sequence along with a donor sequence consisting of the wild type sequence were generated as described in Example 1.5, and a functional test was carried out following electroporation into HEK293L cells. These viral expression vectors (Donor vectors) contained also a Reporter system composed of both mCherry and GFP coding sequences. HEK293 cells were transfected with the Donor vector alone or in combination with the viral expression vector comprising the nucleotide sequence encoding for Cas9 (Cas9 vector). Fluorescent Activated Cells Sorting (FACS) analysis was performed on transfected cells 48 hours post-transfection. In cells transfected with the Donor vector alone, mCherry expression was detected but not GFP expression. Conversely, in cells transfected with both Donor and Cas9 vectors, a proportion of mCherry positive cells also expressed GFP (69.9% and 47.1%, respectively), confirming Cas9 activation. To further confirm vectors expression in patient-specific cells, primary fibroblasts were transfected with the viral expression vectors specific for the c.473C>T (p.(Thr158Met)) MECP2 mutation (Donor vector of SEQ ID NO. 3 and Cas9 vector of SEQ ID NO. 4, respectively) and the c.688C>T (p.(Arg230Cys)) FOXG1 mutation. Fluorescence was visualized in vivo 48 hours post-transfection (40× magnification) and quantified by FACS, confirming the expression of the CRISPR-Cas system also in these cells (FIG. 5).

After expression validation, the present inventors evaluated mutation correction efficiency of the CRISPR-Cas system of the invention in patients' fibroblasts carrying the c.473C>T (p.(Thr158Met))MECP2 mutation or the c.688C>T (p.(Arg230Cys))FOXG1 mutation. Co-transfected mCherry+/GFP+ cells were isolated using BD FACSAria II (BD Biosciences-US). Total DNA was extracted from these cells and analyzed by NGS. The efficiency of genome editing procedure was assessed by Cas-Analyzer tool (http://www.rgenome.net/cas-analyzer/#!) using NGS data and considering a region of 100 nucleotides around the cut site. For FOXG1 c.688C>T (p.(Arg230Cys)) mutation, taking into account that the original cell sample was made of heterozygous cells, NGS results showed a switch in the proportion of the mutant allele from about 50% to 14% following genome editing. Cas-analyzer indicated that 22% of mutant alleles had been successfully edited. For MECP2 c.473C>T (p.(Thr158Met)) mutation, the results from sequencing analysis demonstrated that the proportion of wild type alleles switch from 50% to 70-80% and that the editing was successfully completed in 64% of the alleles.

### Example 2.3: Guide-Seq Analysis for Off-Target Evaluation

To determine the occurrence of off-target events, Guide-seq analysis was performed on HEK293 cells harboring the c.473C>T (p.(Thr158Met))MECP2 mutation. To this aim, cells were co-transfected with the Donor vector of SEQ ID NO. 3 and the Cas9 vector of SEQ ID NO. 4 along with double-stranded oligodeoxynucleotide (dsODN) and the PBML4 plasmid. Following selection of co-transfected cells, DNA was extracted and sequenced on Illumina Miseq instrument. The informatics analysis carried out on sequencing results demonstrated the presence of off-target cuts in less than 1% of the reads. In all cases, aberrant cuts generated by Cas9 were located in non-coding sequences.

### Example 2.4: Correction of COL4 Mutations in Alport Podocytes-Lineage Cells

To confirm that the CRISPR-Cas system of the invention is suitable to be used in Alport Syndrome podocytes-lineage cells, the present inventors verified gene editing correction of specific target mutations in patient-derived cells. Two stable podocyte cell lines carrying the specific mutation c.1871G>A (p.(Gly624Asp)) in COL4A5 gene and the specific mutation c.2567G>A (p.(Gly856Glu)) in COL4A3 gene, respectively, were transfected using the dual vector system of the invention harboring both a Cas9/sgRNA combination for a targeted dsDNA cut and a template Donor DNA for the neo-synthesis of a wild-type DNA fragment. Particularly, the donor vector of SEQ ID NO. 1 and the Cas9 vector of SEQ ID NO. 2 were employed for targeting the COL4A5 c.1871G>A mutation. Also in these experiments, the mCherry/GFP reporter system was used for the identification of the cells wherein the target mutation was corrected. After 48 hours from transfection, the cells were sorted using BD FACSAria II (BD Biosciences-US), to recover the double positive cells (Red/Green fluorescence). Total DNA was extracted from these cells and analyzed by deep sequencing through Ion Torrent S5 (Life Technologies™, Carlsbad, Calif., United States).

Transfected samples and control FASTQ files, which are returned by the sequencing platform, were uploaded to the online analysis tool CasAnalyzer (http://www.rgenome.net/cas-analyzer/#!), in order to obtain the percentage of HDR achieved, considering a comparison range (R) of 50 nucleotides around the cut site.

The results of this analysis showed that the CRISPR-Cas system of the invention is extremely efficient in stably reverting the causative mutation in COL4A5 gene (percentage of 33.2% HDR) and in COL4A3 gene (percentage of 30.4% HDR). For both corrected mutations, the IGV Visualization Software (Broad Institute, Cambridge, United States) was used to visualize both the snip out and the replacement of the mutant base and the indels events erroneously present in the neighboring sites.

### Example 2.5: Mouse Models

Proof-of-principle of central nervous system (CNS) transduction by peripheral injection in Rett syndrome, Pompe and PD mouse models

Since AAV9 is able to cross the blood-brain barrier, the present inventors compared intracranial injection by stereotaxis system with peripheral injection through superficial tail vein. A preliminary experiment conducted in wild-type mice in vivo demonstrated that peripheral administration of AAV9 vectors by intravenous injection of 2×10¹¹ viral genomes (vg) was able to reach central neurons as shown by immunohistochemistry performed after 48 hours. Intravenous injection was performed in 9 months old mice with 5×10¹² vg/kg. In the Pompe disease model, intramuscular injection into the gastrocnemius muscles at birth and at P1 to maximize the amount of injected vector (7×10¹⁰ vg total in 40 μl, 10 μl per GA at P0 and at P1) was compared with muscular injection into both the tricipites and gastrocnemius muscles (7×10¹⁰ vg to 1.6×10¹¹ vg total in 80 μl, 10 μl per muscle at P0 and at P1). The combined injection resulted to be more efficient in achieving both muscular and CNS localization. While direct intracranial injection resulted in a five-fold increase of editing efficiency, peripheral injection by intravenous or intramuscular route was considered sufficient to observe a phenotypic effect.

Proof-of-Principle of In Vivo Correction by Intrarenal Artery Injection in an ATS Dog Model

Building upon the in vitro data, the present inventors set-up a preclinical trial on a naturally occurring ATS dog model. It is known in the art that a 10-base-pair deletion in the COL4A5 gene located on the X chromosome results in the inability to synthesize complete α5 chains giving origin to an X-linked hereditary nephropathy (XLHN) in this dog colony. A single femoral artery catheterization was used to deliver the AAV2-CRISPR/Cas9 system in vivo (Donor vector of SEQ ID NO. 1 and Cas9 vector of SEQ ID NO. 2), injecting into both renal arteries. This procedure was performed for two dosages: 10¹¹-10¹² and 10¹³-10¹⁴. One of the affected dog males was treated with 10¹¹-10¹² of wt-AAV2-CRiSPR/Cas9, one with 10¹³-10¹⁴ of wt-AAV2-CRiSPR/Cas9 and the other with mut-AAV2-CRiSPR/Cas9 as a negative control. In order to assess safety, specificity and efficiency of the viral infection, as well as specificity of CRISPR/Cas9 targeting, dogs were treated at 8 weeks of age, which is typically before the occurrence of microalbuminuria, currently the first clinical indication of disease.

### Example 2.6: Human Studies

Rett Syndrome

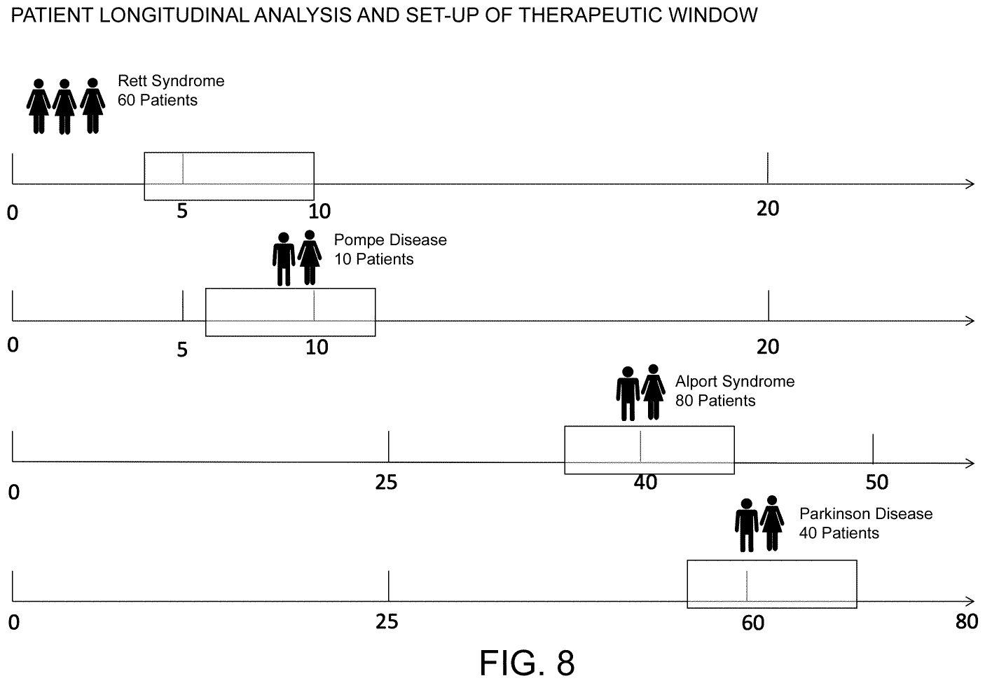

The present inventors operate in an internationally recognised Center of expertise for Rett syndrome and related disorders and therefore have a long lasting experience on the natural history of these disorders, which treatment is presently unavailable. A cohort of 60 patients, for which a follow up every 6 months was available in the last 20 years, were analysed with the specific aim to set-up the most appropriate therapeutic window to be used in a clinical trial. The proposed window (FIG. 8 shows an example for patients carrying MECP2 mutations) takes into account that, although a prompt therapy is essential and desirable, clinical diagnosis is almost impossible up to 3 years and that the patient clinical condition at age of 4 is highly variable and dependent on the type of mutation and rehabilitation treatments.

Pompe Disease

In Pompe disease, current enzyme replacement therapy with recombinant human (rh)GAA has demonstrated efficacy in subjects with late-onset. However, long-term effects of rhGAA on pulmonary function have not been observed, likely related to inefficient delivery of rhGAA to skeletal muscle lysosomes and associated deficits in the central nervous system.

Participants are enrolled with documented GAA enzyme deficiency from blood, skin, or muscle tissue, in which at least one pathogenic mutation has been identified, in a time-window in which participants are still naïve to treatment with α-glucosidase.

Alport Syndrome.

The laboratory of the present inventors is a referral center for diagnosis and research in Alport syndrome. Commonly drug-based approaches in ATS include angiotensin-converting enzyme inhibitor and angiotensin receptor blockers, which are employed to reduce proteinuria and thus retard kidney disease progression and cardiovascular morbidity and mortality. Conventional treatment is commonly started for urinary proteins/urinary creatinine levels above 0.2 mg. CRISPR/Cas9-based gene therapy in ATS disease provides a therapeutic approach acting on the disease-relevant cell lines at the earliest stage of the disease, before the need for conventional therapies.

Women and men were included in the pilot trial in the proportion of half and half in order to study sex-specific drug effects or bias. Children older than 14 years of age were included as well. A cohort of 80 ATS patients, for which a yearly follow up was available in the last 50 years, are analyzed in order to identify individuals with an identified COL4A3 and/or COL4A4 and/or COL4A5 pathogenic mutation, men with X-linked, autosomal dominant, autosomal recessive and digenic inheritance and women with autosomal dominant, autosomal recessive and digenic inheritance between 14 and 60 years of age. This stratified study allows to include women carriers of a COL4A5 mutation who can display disease progression later in life. Patients are observed over a time-window period; male with a COL4A5 mutation and both male and female with a COL4A4 and/or COL4A3 mutation are enrolled when even in the presence of a normal renal function (normal GFR/BSA) microhematuria alone or in combination with podocyturia and/or urinary proteins/urinary creatinine <0.2 mg is observed. COL4A5 mutation carrier females are enrolled in the presence of microhematuria and podocyturia and/or microalbuminuria, indicative of kidney damage progression.

Parkinson's Disease

In Parkinson's disease, when dopaminergic neuronal depletion reaches more than 50%, motor symptoms become evident. Monoamine oxidase-B inhibitors can be considered for initial treatment of early disease. However, levodopa, coupled with carbidopa, remains the gold standard of symptomatic treatment. Unfortunately, its long-term use is associated with motor fluctuations (“wearing-off”) and dyskinesias difficult to treat. Dopamine agonists provide moderate symptomatic benefit and delay the development of dyskinesia compared with levodopa. Symptomatic medications usually prove to be effective for about 4-6 years. After this, disability often progresses despite best medical management, and many patients develop long-term motor complications, including fluctuation, dyskinesias, postural instability and dementia. Thus, therapy for late disease requires different strategies.

LRRK2 is an ideal pharmacological target and in the last few years, an impressive number of LRRK2 kinase inhibitors have been developed. There are, however, important safety issues associated with LRRK2 kinase inhibition, including lung toxicity, likely due to the difficulty in dosing the inhibitor and to its intrinsic off-target effects, as shown in rodents and non-human primates.

Since motor symptoms become evident when dopaminergic neuronal depletion reaches more than 50%, a genome editing approach aiming to restore, even partially, LRRK2/GBA or Parkinson genes before this precise time-window is a challenging strategy in PD treatment. PD patients are thus observed over the time and individuals greater than or equal to 18 years are enrolled in the presence of the following parameters:
1. cardinal signs such as bradykinesia, plus the presence of at least 1 of the following: resting tremor, rigidity, or impairment of postural reflexes, and without any other known or suspected cause of Parkinsonism;
2. a Hoehn & Yahr stage less than or equal to 3;
3. a Mini Mental State Examination (MMSE) score of greater than or equal to 25;
4. Unified Parkinson's Disease Rating Scale (UPDRS) motor score (Part III) of greater than or equal to 10 but less than or equal to 30 at Screening;
5. a DAT scan revealing that a relevant number of dopaminergic neurons has been lost.

Common Inclusion and Exclusion Criteria

On the basis of the preclinical studies results, the present inventors set the following general inclusion and exclusion criteria:

Inclusion Criteria:
age from six months to 6 years old on day of vector infusion for pediatric population;
age from 18 to 70 years for adult cases;
presence of one of the selected mutations.

Exclusion Criteria:
active viral infection (including HIV or serology positive for hepatitis B or C);
any use of invasive ventilatory support or pulse oximetry <95% saturation;
concomitant illness that creates unnecessary risks for gene transfer.
concomitant use of agents used to treat diabetes mellitus or ongoing immunosuppressive therapy or immunosuppressive therapy within 3 months of starting the trial (e.g. corticosteroids, cyclosporine, tacrolimus, methotrexate, cyclophosphamide, intravenous immunoglobulin, rituximab);
patients with anti-AAV antibody titers >1:50 as determined by ELISA binding immunoassay;
abnormal laboratory values considered clinically significant (GGT>3xULN, bilirubin
≥3.0 mg/dL, creatinine ≥1.8 mg/dL, Hgb <8 or >18 g/Dl; WBC >20,000 per cmm);
participation in a recent treatment clinical trial that in the opinion of the PI creates unnecessary risks for gene transfer;
family does not want to disclose patient's study participation with primary care physician and other medical providers.

1. A non-naturally occurring or engineered “Clustered Regularly Interspersed Short Palindromic Repeat (CRISPR)-CRISPR associated (Cas) (CRISPR-Cas)” system targeting a mutant genomic target sequence carrying one or more mutations in a target cell, the system comprising a first viral expression vector and a second viral expression vector, wherein the first viral expression vector comprises:
1) a nucleotide sequence encoding a guide RNA (gRNA) operably linked to a promoter sequence located on the vector upstream of the 5′ end of said nucleotide sequence encoding the gRNA, wherein said gRNA comprises a scaffold nucleotide sequence capable of binding an endonuclease enzyme and a guide nucleotide sequence capable of hybridizing to the mutant genomic target sequence; and
2) a donor nucleotide sequence consisting of the wild type sequence of the mutant genomic target sequence; and
wherein the second viral expression vector comprises:
3) a nucleotide sequence encoding an endonuclease enzyme, said nucleotide sequence being operably linked to a promoter sequence located on the vector upstream of the 5′ end of said nucleotide sequence encoding the endonuclease enzyme; and
4) a target nucleotide sequence consisting of the mutant genomic sequence, said target nucleotide sequence being present on the viral expression vector in one copy or, alternatively, in a first copy and a second copy, wherein, when said target nucleotide sequence is present in one copy, said one copy is located between the 3′ end of the promoter sequence operably linked to the nucleotide sequence encoding the endonuclease enzyme and the 5′ end of said nucleotide sequence encoding the endonuclease enzyme, or, when said target nucleotide sequence is present in a first copy and a second copy, said first copy is located upstream of the 5′ end of the promoter sequence operably linked to the nucleotide sequence encoding the endonuclease enzyme and said second copy is located downstream of the 3′ end of said nucleotide sequence encoding the endonuclease enzyme, each copy of the target nucleotide sequence being flanked at the 3′ end by a Protospacer Adjacent Motif (PAM) sequence.

2. The CRISPR-Cas system according to claim 1, wherein the first viral expression vector and/or the second viral expression vector is an adeno-associated virus (AAV) vector, preferably a serotype 2 adeno-associated viral vector (AAV2) or a serotype 9 adeno-associated viral vector (AAV9).

3. The CRISPR-Cas system according to claim 1 or 2, wherein the endonuclease is a Cas9 endonuclease, preferably a Streptococcus pyogenes Cas9 (SpCas9).

4. The CRISPR-Cas system according to any of claims 1 to 3, wherein the mutant genomic target sequence in the target cell is selected from the group consisting of mutant COL4A5 gene, mutant COL4A3 gene, mutant COL4A4 gene, mutant GAA gene, mutant MECP2 gene, mutant FOXG1 gene, mutant CDKL5 gene, mutant LRRK2 gene, mutant VPS35 gene, mutant PRKN gene, mutant DJ-1 gene, mutant SNCA gene, mutant PINK1 gene and mutant GBA1 gene.

5. A set of two viral particles, wherein one viral particle comprises the first viral expression vector according to any of claims 1 to 4, and the other viral particle comprises the second viral expression vector according to any of claims 1 to 4.

6. An in vitro method for editing a mutant genomic target sequence in a target cell, said method comprising the steps of:
transducing the target cell with a CRISPR-Cas system according to any of claims 1 to 4, or with a set of two viral particles according to claim 5, and
culturing the transduced target cell under suitable conditions for inducing the expression of the endonuclease enzyme and the guide RNA (gRNA) and for obtaining the formation of a macromolecular complex comprising the endonuclease enzyme associated with said gRNA.

7. An isolated target cell which comprises a CRISPR-Cas system according to any of claims 1 to 4.

8. The isolated target cell according to claim 7, which is a human cell, preferably a human cell affected by a genetic disease.

9. A CRISPR-Cas system according to any of claims 1 to 4, or a set of two viral particles according to claim 5 for use as a medicament.

10. The CRISPR-Cas system or the set of two viral particles according to claim 9, for use in the therapeutic treatment of a genetic disease.

11. The CRISPR-Cas system or the set of two viral particles for use according to claim 10, wherein the genetic disease is selected from the group consisting of Alport syndrome, Pompe disease, Rett syndrome and Parkinson's disease.

12. A pharmaceutical composition comprising a CRISPR-Cas system according to any of claims 1 to 4, or a set of two viral particles according to claim 5, and at least one pharmaceutically acceptable vehicle, excipient and/or diluent.

13. The pharmaceutical composition according to claim 12, for use as a medicament.

14. The pharmaceutical composition according to claim 13, for use in the therapeutic treatment of a genetic disease.

15. The pharmaceutical composition according to any of claims 12 to 14, which is in a form suitable for administration via the enteral or parenteral route.
